(19) United States
(12) Patent Application Publication
(10) Pub. No.: US 2023/0173067 A1
(43) Pub. Date: Jun. 8, 2023

(54) METHODS OF TREATING HER2 POSITIVE CANCER WITH TUCATINIB IN COMBINATION WITH TRASTUZUMAB AND AN OXALIPLATIN-BASED CHEMOTHERAPY

(71) Applicant: Seagen Inc., Bothell, WA (US)

(72) Inventor: Luke Walker, Bothell, WA (US)

(21) Appl. No.: 17/926,206

(22) PCT Filed: May 28, 2021
(86) PCT No.: PCT/US2021/034715

§ 371 Date: Nov. 18, 2022

Related U.S. Application Data
(60) Provisional application No. 63/032,223, filed on May 29, 2020.

(51) Int. Cl. A61K 39/395; A61K 31/517; A61K 31/519; A61K 31/513; A61K 31/282; A61K 45/06; A61P 35/00; A61P 1/00; A61P 11/00

(52) U.S. Cl. CPC A61K 39/3955; A61K 31/517; A61K 31/519; A61K 31/513; A61K 31/282; A61K 45/06; A61P 35/00; A61P 1/00; A61P 11/00

(57) ABSTRACT

The present disclosure relates to a method of treating a HER2 positive cancer in a subject in need thereof, the method comprising: administering to the subject a therapeutically effective amount of a combination therapy comprising tucatinib, trastuzumab, and an oxaliplatin-based chemotherapy.

### BACKGROUND

Encoded by the ERBB2 gene, human epidermal growth factor receptor 2 (HER2) is part of a family of 4 related receptor tyrosine kinases, which include HER1 (also known as epidermal growth factor receptor [EGFR]), HER2, HER3, and HER4. HER1-4 are single-pass transmembrane glycoprotein receptors containing an extracellular ligand binding region and an intracellular signaling domain. HER2 has no known ligand, but it is the preferred dimerization partner for the other HER family receptors. When overexpressed in tumors, HER2 forms ligand-independent homodimeric complexes that autophosphorylate. HER2 homo- or heterodimerization results in the activation of multiple signaling cascades, including the Ras/Raf/MEK/MAPK, PI3K/AKT, Src, and STAT pathways. These signaling pathways lead to cell proliferation, inhibition of apoptosis, and metastasis.

The treatment and prevention of HER2 positive cancers represents an unmet need. Cancers that are characterized by the overexpression of HER2 (referred to as HER2 positive cancers) are often correlated with poor prognosis or are resistant to many standard therapies. Accordingly, there is a need for new therapies that are effective for the treatment of cancers such as HER2 positive cancers or metastatic HER2 positive cancers.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating cancer in a subject in need thereof, the methods comprising identifying the subject as having a HER2 positive cancer; and administering to the subject a therapeutically effective amount of a combination therapy comprising tucatinib, trastuzumab, and an oxaliplatin-based chemotherapy. Also provided herein are methods of treating a HER2 positive cancer in a subject in need thereof, the method comprising administering to the subject a therapeutically effective amount of a combination therapy comprising tucatinib, trastuzumab and an oxaliplatin-based chemotherapy.

In some embodiments of any of the methods described herein, the oxaliplatin-based chemotherapy is oxaliplatin in combination with a compound selected from the group consisting of leucovorin (LV), fluorouracil, uracil-tegafur (UFT), irinotecan, and bevacizumab, or a combination thereof.

In some embodiments of any of the methods described herein, the oxaliplatin-based chemotherapy is administered as a regimen selected from the group consisting of FOLFOX4, mFOLFOX4, FOLFOX6, mFOLFOX6, FOLFOX7, mFOLFOX7, FOLFOXIRI, bFOL, PVIFOX, IROX, FUOX, FuFOX, CapeOx, XELOX, and CAPOX.

In some embodiments of any of the methods described herein, the oxaliplatin-based chemotherapy comprises administering oxaliplatin, leucovorin, and fluorouracil by an mFOLFOX7 regimen.

In some embodiments of any of the methods described herein, the HER2 positive cancer is selected from the group consisting of gastric adenocarcinoma, gastroesophageal junction (GEJ) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, brain cancer, and breast cancer.

In some embodiments of any of the methods described herein, the HER2 positive cancer is gastric adenocarcinoma, gastroesophageal junction (GEJ) adenocarcinoma, esophageal adenocarcinoma, colorectal carcinoma (CRC), cholangiocarcinoma, or gallbladder carcinoma. In some embodiments, the HER2 positive cancer is unresectable or metastatic.

In some embodiments of any of the methods described herein, the subject has not been previously treated with a cancer therapy. In some embodiments, of any of the methods described herein, the subject has not been previously treated with tucatinib. In some embodiments of any of the methods described herein, the subject has not been previously treated with trastuzumab. In some embodiments of any of the methods described herein, the subject has not been previously treated with an oxaliplatin-based chemotherapy.

In some embodiments of any of the methods described herein, the subject is a candidate to receive an oxaliplatin-based chemotherapy.

In some embodiments of any of the methods described herein, the subject was previously treated or is a caudate to receive an anticancer therapy. In some embodiments of any of the methods described herein, the subject was previously treated or is currently being treated with an oxaliplatin-based chemotherapy. In some embodiments of any of the methods described herein, the subject was previously treated with at least one anticancer therapy. In some embodiments of any of the methods described herein, the least one anticancer therapy is an anti-HER2 antibody or anti-HER2 antibody-drug conjugate. In some embodiments of any of the methods described herein, the at least one previous anticancer therapy is selected from the group consisting of trastuzumab, trastuzumab and a taxane, pertuzumab, ado-trastuzumab (T-DM1), and combinations thereof.

In some embodiments of any of the methods described herein, the subject is refractory to the previous anticancer therapy. In some embodiments of any of the methods described herein, the subject developed a brain metastasis during the previous anticancer therapy.

In some embodiments of any of the methods described herein, the subject has not been treated with another anticancer therapy within the past 12 months.

In some embodiments of any of the methods described herein, the tucatinib is administered to the subject at a dose of about 100 mg to about 1000 mg. In some embodiments of any of the methods described herein, the tucatinib is administered twice daily. In some embodiments of any of the methods described herein, the tucatinib is administered to the subject orally. In some embodiments of any of the methods described herein, the trastuzumab is administered to the subject at a dose of about 6 mg/kg. In some embodiments of any of the methods described herein, the trastuzumab is administered to the subject at a dose of about 4 mg/kg.

Also provided herein are methods for treating a HER2 positive cancer in a subject that has exhibited an adverse event after starting treatment with a combination therapy comprising tucatinib, trastuzumab, and an oxaliplatin-based chemotherapy at an initial dosage level, comprising administering to the subject the at least one component of the combination therapy at a reduced dosage level.

In some embodiments of any of the methods described herein, the tucatinib is administered to the subject at a dose of about 100 mg to about 1000 mg. In some embodiments of any of the methods described herein, the trastuzumab is administered to the subject at a dose of about 6 mg/kg. In some embodiments of any of the methods described herein, the trastuzumab is administered to the subject at a dose of about 4 mg/kg.

Also provided herein are kits for treating or ameliorating the effects of a HER2 positive cancer in a subject, the kit comprising tucatinib, trastuzumab, and an oxaliplatin-based chemotherapy.

Also provided herein are methods of treating a HER2 positive cancer in a subject in need thereof, the methods comprising administering to the subject a therapeutically effective amount of a combination therapy comprising tucatinib, trastuzumab, and an oxaliplatin-based chemotherapy, and administering an effective amount of an anti-diarrheal agent.

Also provided herein are methods of reducing the severity or incidents of diarrhea, or preventing diarrhea in a subject having a HER2 positive cancer and being treated with an effective amount of a combination therapy comprising tucatinib, trastuzumab, and an oxaliplatin-based chemotherapy, the methods comprising administering an effective amount of an anti-diarrheal agent prophylactically.

Also provided herein are methods of reducing the likelihood of a subject developing diarrhea, wherein the subject has a HER2 positive cancer and is being treated with an effective amount of a combination therapy comprising tucatinib, trastuzumab, and an oxaliplatin-based chemotherapy the method comprising administering an effective amount of an anti-diarrheal agent prophylactically.

In some embodiments of any of the methods described herein, the combination therapy and the anti-diarrheal agent are administered concurrently. In some embodiments of any of the methods described herein, the anti-diarrheal agent is administered prior to administration of the combination therapy. In some embodiments of any of the methods described herein, the subject is exhibiting symptoms of diarrhea. In some embodiments of any of the methods described herein, the subject is not exhibiting symptoms of diarrhea.

### DESCRIPTION OF THE DRAWINGS

FIG. 1 Shows a dose escalation cohort associated with a Phase 1b, dose escalation study of tucatinib in combination with trastuzumab and an oxaliplatin-based chemotherapy for HER2+ positive cancers described herein in connection with Example 1.

FIG. 2 provides the amino acid sequence of the heavy (SEQ. ID NO. 1) and light chains (SEQ. ID NO. 2) of trastuzumab and the light chain variable domain (SEQ. ID NO. 3) and the heavy chain variable domain (SEQ. ID NO. 4).

FIGS. 3A-3C show that a combination of tucatinib and trastuzumab was active in HER2 amplified colorectal cancer (CRC) patient-derived xenograft (PDX) models. Data are shown as group mean+/−S.E.M. FIG. 3A shows the effects of tucatinib and trastuzumab, alone and in combination, on tumor growth in a CTG-0121 CRC PDX model. FIG. 3B shows the effects of tucatinib and trastuzumab, alone and in combination, on tumor growth in a CTG-0784 CRC PDX model. FIG. 3C shows the effects of tucatinib and trastuzumab, alone and in combination, on tumor growth in a CTG-0383 CRC PDX model.

FIGS. 4A and 4B show that a combination of tucatinib and trastuzumab was active in HER2 amplified esophageal cancer patient-derived xenograft (PDX) models. Data are shown as group mean+/−S.E.M. FIG. 4A shows the effects of tucatinib and trastuzumab, alone and in combination, on tumor growth in a CTG-0137 esophageal cancer PDX model. FIG. 4B shows the effects of tucatinib and trastuzumab, alone and in combination, on tumor growth in a CTG-0138 esophageal cancer PDX model.

FIGS. 5A-5C show that a combination of tucatinib and trastuzumab was active in HER2 positive gastric cancer patient-derived xenograft (PDX) models. Data are shown as group mean+/−S.D. FIG. 5A shows the effects of tucatinib and trastuzumab, alone and in combination, on tumor growth in a GXA 3038 gastric cancer PDX model. FIG. 5B shows the effects of tucatinib and trastuzumab, alone and in combination, on tumor growth in a GXA 3039 gastric cancer PDX model. FIG. 5C shows the effects of tucatinib and trastuzumab, alone and in combination, on tumor growth in a GXA 3054 gastric cancer PDX model.

FIG. 6 shows that a combination of tucatinib and trastuzumab was active in a CTG-0927 HER2 positive cholangiocarcinoma patient-derived xenograft (PDX) model. Data are shown as mean+/−S.E.M.

FIGS. 7A and 7B show that a combination of tucatinib and trastuzumab was active in HER2 positive non-small cell lung cancer (NSCLC) models. Data are shown as group mean+/−S.E.M. FIG. 7A shows the effects of tucatinib and trastuzumab, alone and in combination, on tumor growth in a Calu-3 NSCLC xenograft model. FIG. 7B shows the effects of tucatinib and trastuzumab, alone and in combination, on tumor growth in an NCI-H2170 NSCLC xenograft model.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the disclosure described herein include “comprising,” “consisting,” and “consisting essentially of” aspects and embodiments. It is understood that aspects and variations of the embodiments described herein include “consisting of” and/or “consisting essentially of” aspects and variations. In some embodiments, methods consisting essentially of an administration step as disclosed herein include methods wherein a patient has failed a prior therapy (administered to the patient before the period of time) or has been refractory to such prior therapy, and/or wherein the cancer has metastasized or recurred. In some embodiments, methods consisting essentially of an administration step as disclosed herein include methods wherein a patient undergoes surgery, radiation, and/or other regimens prior to, substantially at the same time as, or following such an administration step as disclosed herein, and/or where the patient is administered other chemical and/or biological therapeutic agents following such an administration step as disclosed herein.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

As used herein, the term “co-administering” includes sequential or simultaneous administration of tucatinib, trastuzumab, and an oxaliplatin-based chemotherapy. For example, the co-administered compounds can be administered by the same route. In other instances, the co-administered compounds are administered via different routes. For example, one or two compounds can be administered orally, and the other compound(s) can be administered, e.g., sequentially or simultaneously, via intravenous, intramuscular, subcutaneous, or intraperitoneal injection. The simultaneously or sequentially administered compounds or compositions can be administered such that the trastuzumab and/or the oxaliplatin-based chemotherapy and tucatinib are simultaneously present in a subject or in a cell at an effective concentration.

As used herein, the term “combination”, “therapeutic combination”, “combination therapy”, or “pharmaceutical combination”, as used herein, defines either a fixed combination in one dosage unit form or a kit of parts or instructions for the combined administration where the tucatinib, the trastuzumab, and the oxaliplatin-based chemotherapy may be administered independently at the same time or separately within time intervals that allow that the combination partners show a cooperative, e.g., synergistic, effect.

A “cancer” refers to a broad group of various diseases characterized by the uncontrolled growth of abnormal cells in the body. A “cancer” or “cancer tissue” can include a tumor.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging can be used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “TO” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “NO” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “M0” denotes that no metastasis has occurred, and “M1” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, gastric adenocarcinoma, gastroesophageal junction (GEJ) adenocarcinoma, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP_001289938, and NP_004448. Non-limiting examples of HER2 peptide sequences are set forth in GenBank reference numbers NP_001005862, NP_001276865, NP_001276866, NP_001276867, and NP_004439. Each of these sequences are hereby incorporated by reference in their entireties.

When HER2 is amplified or overexpressed in or on a cell, the cell is referred to as being “HER2 positive.” The level of HER2 amplification or overexpression in HER2 positive cells is commonly expressed as a score ranging from 0 to 3 (i.e., HER2 0, HER2 1+, HER2 2+, or HER2 3+), with higher scores corresponding to greater degrees of expression.

The term “HER2 positive-associated” with respect to a disease or disorder, as used herein refers to diseases or disorders associated with amplification or overexpression of HER2. Non-limiting examples of HER2 positive-associated diseases or disorders can include, for example, HER2 positive breast cancer (e.g., “HER2 positive breast cancer-associated”).

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “metastatic cancer” (also known as “secondary cancer”) as used herein refers to a type of cancer that originates in one tissue type, but then spreads to one or more tissues outside of the (primary) cancer's origin. Following metastasis, the distal tumors can be said to be “derived from” the pre-metastasis tumor. For example, a “tumor derived from” a breast cancer refers to a tumor that can be the result of a metastasized breast cancer. Metastatic brain cancer refers to cancer in the brain, i.e., cancer which originated in a tissue other than the brain and has metastasized to the brain.

The term “tucatinib,” also known as ONT-380, or ARRY-380, refers to the small molecule tyrosine kinase inhibitor that suppresses or blocks HER2 activation having the following structure:

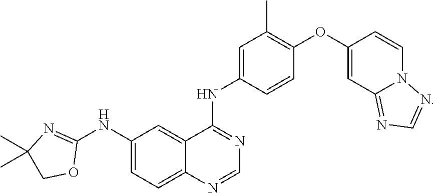 [SMILES: Cc1cc(Nc2ncnc3ccc(NC4=NC(C)(C)CO4)cc23)ccc1Oc1ccn2ncnc2c1]

In some instances, tucatinib can be in the form of a pharmaceutically acceptable salt.

The term “oxaliplatin-based chemotherapy” refers to administration of oxaliplatin or administration of a combination chemotherapy treatment comprising oxaliplatin. For example, an oxaliplatin-based chemotherapy can include oxaliplatin in combination with other chemotherapeutics such as leucovorin (LV) (folinic acid), or a pharmaceutically acceptable salt thereof such as leucovorin calcium (calcium folinate), fluorouracil (e.g., 5-fluorouracil e.g., 5-FU), uracil-tegafur (UFT), irinotecan, and bevacizumab (see, e.g., Rosati et al. Cancers 2019, 11, 578; doi:10.3390/cancers11040578, and Yaffee et al. Journal of Gastrointestional Oncology 2015, 6 (2): 185-200. doi: 10.3978/j.issn. 2078-6891.2014.112 hereby incorporated by reference in their entirety for all purposes). An oxaliplatin-based chemotherapy can be administered using a particular regimen of the oxaliplatin in combination with other chemotherapeutics. In some embodiments, an oxaliplatin-based chemotherapy consists of administration of oxaliplatin.

Non-limiting examples of oxaliplatin-based chemotherapy regimens include FOLFOX4 (oxaliplatin 85 mg/m², LV 200 mg/m²/day or 1-LV 100 mg/m²/day, and fluorouracil bolus 400 mg/m²/day followed by continuous infusion 600 mg/m²/day), modified FOLFOX4 (mFOLFOX4) (oxaliplatin 85 mg/m² IV infusion, LV 50 mg IV bolus, fluorouracil 400 mg/m² IV, and fluorouracil 1,200 mg/m² CIV via pump), FOLFOX6 (oxaliplatin 100 mg/m², LV 400 mg/m², fluorouracil 400 mg/m² given as an intravenous bolus injection and then fluorouracil 2,400-3,000 mg/m² as a 46-hour civ), modified FOLFOX6 (mFOLFOX6) (oxaliplatin 100 mg/m², leucovorin 400 mg/m², and fluorouracil 400 mg/m²), FOLFOX7 (oxaliplatin 130 mg/m², LV 400 mg/m², fluorouracil, 400 mg/m²), modified FOLFOX7 (mFOLFOX7) (oxaliplatin 85 mg/m², leucovorin 200 mg/m², and fluorouracil 2400 mg/m²), FOLFOXIRI (irinotecan 165 mg/m² IV, oxaliplatin 85 mg/m² IV, LV 400 mg/m², fluorouracil 1,600 mg/m²/day×2 days (total 3,200 mg/m² over 48 hours), bFOL (oxaliplatin 85 mg/m² IV, LV 20 mg/m² IV, fluorouracil 500 mg/m² IV), PVIFOX (dexamethasone 20 mg, granisetron 3 mg, oxaliplatin 130 mg/m², fluorouracil 250 mg/m²/daily), IROX (irinotecan 150 mg/m², and oxaliplatin 85 mg/m²), FUOX (oxaliplatin 85 mg/m², folinic acid 2250 mg/m², fluorouracil 2000 mg/m²), FuFOX (fluorouracil 2,250 mg/m², oxaliplatin 85 mg/m²), CapeOx (oxaliplatin 130 mg/m², capecitabine 1000 mg/m²), XELOX (oxaliplatin 130 mg/m², capecitabine 1000 mg/m²), and CAPOX (oxaliplatin 70 mg/m², capecitabine 1,000 mg/m²).

In some embodiments, the oxaliplatin-based chemotherapy is administered as a regimen selected from the group consisting of FOLFOX4, mFOLFOX4, FOLFOX6, mFOLFOX6, FOLFOX7, mFOLFOX7, FOLFOXIRI, bFOL, PVIFOX, IROX, FUOX, FuFOX, CapeOx, XELOX, and CAPOX.

In some embodiments, mFOLFOX7 regimen comprises oxaliplatin 85 mg/m², leucovorin 200 mg/m², and fluorouracil 2400 mg/m² which is administered by IV every 2 weeks starting from Cycle 1 Day 1 onwards where the fluorouracil is administered as a continuous infusion.

The term “anti-HER2 antibody-drug conjugate” refers to an anti-HER2 antibody conjugated to a therapeutic agent (i.e., a drug) optionally via a linker.

An “anti-HER2 antibody”, as used herein, refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include trastuzumab, pertuzumab, margetuximab, and combinations thereof.

The term “ado-trastuzumab emtansine”, also known as T-DM1, refers to an antibody-drug conjugate composed of trastuzumab, a thioether linker, and a derivative of the antimitotic agent maytansine (also known as DM1). Ado-trastuzumab emtansine is sold in the U.S. under the trade name KADCYCLA®. As used herein, “ado-trastuzumab emtansine” also includes biosimilars of trastuzumab, for example, Kanjinti (trastuzumab-anns).

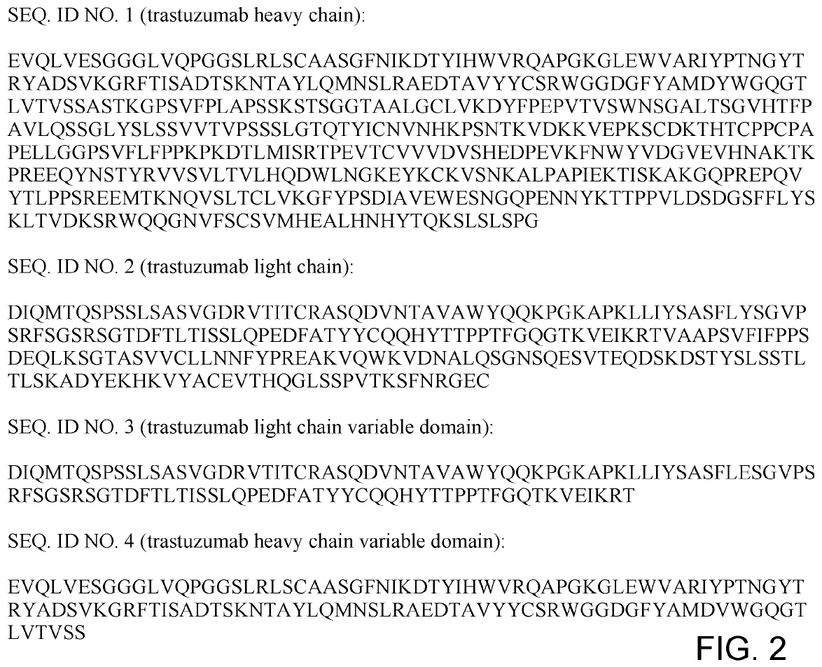

A “biosimilar” as used herein refers to an antibody or antigen-binding fragment that has the same primary amino acid sequence as compared to a reference antibody (e.g., trastuzumab) and optionally, may have detectable differences in post-translation modifications (e.g., glycosylation and/or phosphorylation) as compared to the reference antibody (e.g., a different glycoform). As a reference, the amino acid sequence of the heavy chain of trastuzumab is provided as SEQ. ID NO. 1, the light chain of trastuzumab is provided as SEQ. ID NO. 2, the light chain variable domain (SEQ. ID NO. 3), and the heavy chain variable domain (SEQ. ID NO. 4) (see also FIG. 2 and U.S. Pat. No. 5,821,337, which is incorporated herein in its entirety).

In some embodiments, a biosimilar is an antibody or antigen-binding fragment thereof that has a light chain that has the same primary amino acid sequence as compared to a reference antibody (e.g., trastuzumab) and a heavy chain that has the same primary amino acid sequence as compared to the reference antibody. In some examples, a biosimilar is an antibody or antigen-binding fragment thereof that has a light chain that includes the same light chain variable domain sequence as a reference antibody (e.g., trastuzumab) and a heavy chain that includes the same heavy chain variable domain sequence as a reference antibody. In some embodiments, a biosimilar can have a similar glycosylation pattern as compared to the reference antibody (e.g., trastuzumab). In other embodiments, a biosimilar can have a different glycosylation pattern as compared to the reference antibody (e.g., trastuzumab).

The term “tumor growth inhibition (TGI) index” refers to a value used to represent the degree to which an agent (e.g., tucatinib, trastuzumab, and oxaliplatin-based chemotherapy or a combination thereof) inhibits the growth of a tumor when compared to an untreated control. The TGI index is calculated for a particular time point (e.g., a specific number of days into an experiment or clinical trial) according to the following formula:

$$TGI = 1 - ( \frac{Volum {e}_{t r e a t ed ( TxDayX )} - Volum {e}_{treate d ( TxDay 0 )}}{Volum {e}_{c o n t r ol ( TxDayX )} - Volum {e}_{c o n t r o l ( TxDay 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

As used herein, the term “synergistic” or “synergy” refers to a result that is observed when administering a combination of components or agents (e.g., a combination of tucatinib, trastuzumab, and oxaliplatin-based chemotherapy) produces an effect (e.g., inhibition of tumor growth, prolongation of survival time) that is greater than the effect that would be expected based on the additive properties or effects of the individual components. In some embodiments, synergism is determined by performing a Bliss analysis (see, e.g., Foucquier et al. Pharmacol. Res. Perspect. (2015) 3(3):e00149; hereby incorporated by reference in its entirety for all purposes). The Bliss Independence model assumes that drug effects are outcomes of probabilistic processes, and asumes that the drugs act completely independently (i.e., the drugs do not interfere with one another (e.g., the drugs have different sites of action) but each contributes to a common result).

The observed effect of a combination of drugs can be based on, for example, the TGI index, tumor size (e.g., volume, mass), an absolute change in tumor size (e.g., volume, mass) between two or more time points (e.g., between the first day a treatment is adminstered and a particular number of days after treatment is first administered), the rate of change of tumor size (e.g., volume, mass) between two or more time points (e.g., between the first day a treatment is adminstered and a particular number of days after treatment is first administered), or the survival time of a subject or a population of subjects. When the TGI index is taken as a measure of the observed effect of a combination of drugs, the TGI index can be determined at one or more time points. When the TGI index is determined at two or more time points, in some instances the mean or median value of the multiple TGI indices can be used as a measure of the observed effect. Furthermore, the TGI index can be determined in a single subject or a population of subjects. When the TGI index is determined in a population, the mean or median TGI index in the population (e.g., at one or more time points) can be used as a measure of the observed effect. When tumor size or the rate of tumor growth is used as a measure of the observed effect, the tumor size or rate of tumor growth can be measured in a subject or a population of subjects. In some instances, the mean or median tumor size or rate of tumor growth is determined for a subject at two or more time points, or among a population of subjects at one or more time points. When survival time is measured in a population, the mean or median survival time can be used as a measure of the observed effect.

When TGI indices are taken as a measure of the observed effects, the TGI indices can be determined at one or more time points. When TGI indices are determined at two or more time points, in some instances the mean or median values can be used as measures of the observed effects. Furthermore, the TGI indices can be determined in a single subject or a population of subjects in each treatment group. When the TGI indices are determined in populations of subjects, the mean or median TGI indices in each population (e.g., at one or more time points) can be used as measures of the observed effects. When tumor sizes or the rates of tumor growth are used as measures of the observed effects, the tumor sizes or rates of tumor growth can be measured in a subject or a population of subjects in each treatment group. In some instances, the mean or median tumor sizes or rates of tumor growth are determined for subjects at two or more time points, or among populations of subjects at one or more time points. When survival time is measured in a population, mean or median survival times can be used as measures of the observed effects.

In some embodiments, a combination of tucatinib, trastuzumab, and an oxaliplatin-based chemotherapy is considered to be synergistic when the combination produces an observed TGI index that is greater than the predicted TGI index for the combination of drugs (e.g., when the predicted TGI index is based upon the assumption that the drugs produced a combined effect that is additive). In some instances, the combination is considered to be synergistic when the observed TGI index is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the predicted TGI index for the combination of drugs.

In some embodiments, the rate of tumor growth (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In other embodiments, survival time is used to determine whether a combination of drugs is synergistic (e.g., a combination of drugs is synergistic when the survival time of a subject or population of subjects is longer than would be expected if the combination of drugs produced an additive effect).

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease.

The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect (e.g., diarrhea).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression (e.g., brain metastasis regression) can be observed and continue for a period of at least about 20 days, at least about 30 days, at least about 40 days, at least about 50 days, or at least about 60 days.

As used herein, “subtherapeutic dose” means a dose of a therapeutic compound (e.g., tucatinib) that is lower than the usual or typical dose of the therapeutic compound when administered alone for the treatment of a hyperproliferative disease (e.g., cancer).

“Simultaneous administration,” as used herein, means that the two or more therapies (e.g., in a combination therapy) are administered with a time separation of no more than about 15 minutes, such as no more than about any of 10, 5, or 1 minutes. When the two or more therapies are administered simultaneously, the two or more therapies can be contained in the same composition (e.g., a composition comprising both a first and second therapy) or in separate compositions (e.g., a first therapy in one composition and a second therapy is contained in another composition).

As used herein, the term “sequential administration” means that the two or more therapies (e.g., in a combination therapy) are administered with a time separation of more than about 15 minutes, such as more than about any of 20, 30, 40, 50, 60, or more minutes. Any of the two or more therapies may be administered first. The two or more therapies are contained in separate compositions, which may be contained in the same or different packages or kits.

As used herein, the term “concurrent administration” means that the administration of two or more therapies (e.g., in a combination therapy) overlap with each other. For example, the two or more therapies can be administered in the same day, or with a time separation of within one day, within two days, within three days, within four days, within five days, within six days, within seven days, within ten days, within fourteen days, or within twenty-one days.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size can remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., breast cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 3.6 mg/kg of an agent, such as tucatinib, trastuzumab, and/or an oxaliplatin-based chemotherapy, one can calculate and use the appropriate amount of the agent (i.e., 216 mg) for administration to said subject.

The use of the term “fixed dose” with regard to a method of the disclosure means that two or more different agents (e.g., tucatinib, trastuzumab, and/or an oxaliplatin-based chemotherapy) are administered to a subject in particular (fixed) ratios with each other. In some embodiments, the fixed dose is based on the amount (e.g., mg) of the agents. In certain embodiments, the fixed dose is based on the concentration (e.g., mg/ml) of the agents.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., tucatinib, trastuzumab, and/or an oxaliplatin-based chemotherapy). For example, a subject with 60 kg body weight and a subject with 100 kg body weight can receive the same dose of tucatinib (e.g., 300 mg).

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The phrase “pharmaceutically acceptable salt” as used herein, refers to pharmaceutically acceptable organic or inorganic salts of a compound of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate (mesylate), ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

As used herein, the term “solid dispersion” means a system in a solid state comprising at least two components, wherein one component is dispersed throughout the other component. For example, a solid dispersion as described herein can include one component of tucatinib dispersed throughout another component, such as a dispersion polymer.

As used herein, the term “amorphous” means a solid in a solid state that is a non-crystalline state. Amorphous solids generally possess crystal-like short range molecular arrangement, but no long range order of molecular packing as found in crystalline solids. The solid state form of a solid may be determined by polarized light microscopy, X-ray powder diffraction (“XRPD”), differential scanning calorimetry (“DSC”), or other standard techniques known to those of skill in the art.

As used herein, the term “amorphous solid dispersion” means a solid comprising a drug substance and a dispersion polymer. The amorphous solid dispersion discussed herein comprises amorphous tucatinib and a dispersion polymer, wherein the amorphous solid dispersion contains tucatinib in a substantially amorphous solid state form. In certain embodiments, the substantially amorphous solid state form means that the tucatinib component in the amorphous solid dispersion is at least 80% amorphous tucatinib. In certain embodiments, the substantially amorphous solid state form means that the tucatinib component in the amorphous solid dispersion is at least 85% amorphous tucatinib. In certain embodiments, the substantially amorphous solid state form means that the tucatinib component in the amorphous solid dispersion is at least 90% tucatinib. In certain embodiments, the substantially amorphous solid state form means that the tucatinib component in the amorphous solid dispersion is at least 95% amorphous tucatinib.

As used herein, the term “dispersion polymer” means a polymer that allows for tucatinib to be dispersed throughout such that a solid dispersion may form. The dispersion polymer is preferably neutral or basic. The dispersion polymer may contain a mixture of two or more polymers. Examples of dispersion polymers include, but are not limited to, vinyl polymers and copolymers, vinylpyrrolidine vinylacetate copolymer (“PVP-VA”), polyvinyl alcohols, polyvinyl alcohol polyvinyl acetate copolymers, polyvinyl pyrrolidine (“PVP”), acrylate and methacrylate copolymers, methylacrylic acid methyl methacrylate copolymer (such as Eudragit®), polyethylene polyvinyl alcohol copolymers, polyoxyethylene-polyoxypropylene block copolymers (also referred to as poloxamers), graft copolymer comprised of polyethylene glycol, polyvinyl caprolactam and polyvinyl acetate (such as Soluplus®), cellulosic polymers, such as hydroxypropyl methyl cellulose acetate (“HPMCA”), hydroxypropyl methyl cellulose (“HPMC”), hydroxypropyl cellulose (“HPC”), methyl cellulose, hydroxyethyl methyl cellulose, hydroxyethyl cellulose, hydroxyethyl cellulose acetate, and hydroxyethyl ethyl cellulose, hydroxypropyl methyl cellulose acetate succinate (“HPMCAS”), hydroxypropyl methyl cellulose phthalate (“HPMCP”), carboxymethylethyl cellulose (“CMEC”), cellulose acetate phthalate (“CAP”), cellulose acetate succinate (“CAS”), hydroxypropyl methyl cellulose acetate phthalate (“HPMCAP”), cellulose acetate trimellitate (“CAT”), hydroxypropyl methyl cellulose acetate trimellitate (“HPMCAT”), and carboxymethylcellulose acetate butyrate (“CMCAB”), and the like.

As used herein, the term “spray drying” means processes involved in breaking up liquid mixtures into small droplets (atomization) and rapidly removing solvent from the mixture in a spray drying apparatus where there is a strong driving force for evaporation of solvent from the droplets. The phrase spray drying is used conventionally and broadly. Spray drying processes and spray drying equipment are described generally in Perry, Robert H., and Don W. Green (eds.). Perry's Chemical Engineers' Handbook. New York: McGraw-Hill, 2007 (8^{th} edition).

As used herein, “polymorphs” refer to distinct solids sharing the same molecular formula, yet each polymorph may have distinct solid state physical properties. A single compound may give rise to a variety of polymorphic forms where each form has different and distinct solid state physical properties, such as different solubility profiles, melting point temperatures, flowability, dissolution rates and/or different X-ray diffraction peaks. These practical physical characteristics are influenced by the conformation and orientation of molecules in the unit cell, which defines a particular polymorphic form of a substance. Polymorphic forms of a compound can be distinguished in a laboratory by X-ray diffraction spectroscopy, such as X-ray powder diffraction (“XRPD”), and by other methods, such as infrared spectrometry. Additionally, polymorphic forms of the same drug substance or active pharmaceutical ingredient can be administered by itself or formulated as a drug product (pharmaceutical composition) and are well known in the pharmaceutical art to affect, for example, the solubility, stability, flowability, tractability and compressibility of drug substances and the safety and efficacy of drug products. For more, see Hilfiker, Rolf (ed.), Polymorphism in the Pharmaceutical Industry. Weinheim, Germany: Wiley-VCH 2006.

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom of a disease contemplated herein (e.g., breast cancer). The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Description of the Embodiments

A. Methods for Treating Cancer with Tucatinib in Combination with Trastuzumab and an Oxaliplatin-Based Chemotherapy

HER2 is a validated target in multiple solid tumors, with anti-HER2 biologics and small-molecule drugs approved for patients with HER2+ breast and gastric cancers. Amplification of the HER2-gene or overexpression of its protein occurs in approximately 15% to 20% of breast cancers and 6% to 30% of gastric and esophageal cancers. Recently, interest has grown in HER2-targeting strategies for patients with refractory metastatic colorectal carcinoma (CRC), where overexpression of HER2 has been found to occur in approximately 3% to 5% of patients. HER2 can also be overexpressed in other gastrointestinal cancers, such as cholangiocarcinoma and gallbladder carcinoma, where studies suggest ERBB2 amplification ranges from 1% to 6%.

The current standard of care for patients with HER2+ metastatic disease consists of treatment with pertuzumab plus trastuzumab and a taxane as first-line treatment for metastatic disease, followed by T-DM1 in second line. Treatment options for patients who progress after treatment with both pertuzumab and T-DM1 remain relatively limited. Patients are generally treated with a continuation of anti-HER2 therapy (in the form of trastuzumab or lapatinib) in combination with cytotoxic chemotherapy, such as capecitabine. Combined HER2 therapy with trastuzumab and lapatinib can also be considered. In some HER2 positive gastrointestional cancers, the standard of care of treatment with trastuzumab and chemotherapy, while treatment with lapatinib has been relatively ineffective against gastrointestional cancers. In other HER2 positive cancers such as gastroesophageal, colorectal, biliary tract, and gallbladder cancers, the standard of care is an oxaliplatin-based chemotherapy comprising the combination of oxaliplatin, fluorouracil, and leucovorin (e.g., FOLFOX and/or a modified FOLFOX regimen). However, no single regimen is considered the standard of care in this setting and better options for these patients are needed. The treatment and prevention of HER2 positive cancers represents an unmet need.

In some aspects, the disclosure provides a method of treating a HER2 positive cancer in a subject in need thereof, the method comprising administering to the subject a therapeutically effective amount of a combination therapy comprising tucatinib, trastuzumab and an oxaliplatin-based chemotherapy.

In some aspects, the disclosure provides a method of treating cancer in a subject in need thereof comprising, identifying the subject as having a HER2 positive cancer. For example, the subject can have a HER2 positive cancer that is histologically or cytologically confirmed. In some embodiments, the HER2 positive cancer is selected from the group consisting of gastric adenocarcinoma, gastroesophageal junction (GEJ) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, brain cancer, and breast cancer. In some embodiments, the HER2 positive cancer is unresectable or metastatic. In some embodiments, the method can further include administering to the subject a therapeutically effective amount of a combination therapy comprising tucatinib, trastuzumab, and an oxaliplatin-based chemotherapy.

In some embodiments, the oxaliplatin-based chemotherapy is oxaliplatin in combination with a compound selected from the group consisting of leucovorin, fluorouracil, uracil-tegafur (UFT), folinic acid, and bevacizumab. In some embodiments, the method comprises administering oxaliplatin, leucovorin, and fluorouracil by an mFOLFOX7 regimen. For example, the mFOLFOX7 regimen includes oxaliplatin 85 mg/m², leucovorin 200 mg/m², and fluorouracil 2400 mg/m². In some examples, the subject should be receiving, or is eligible to receive, an oxaliplatin-based regimen.

In some embodiments, the subject has not been previously treated. For instance, in some embodiments, the subject has not been treated with another anticancer therapy within the past 12 months. In some embodiments, the subject may have not been previously treated with tucatinib. In some embodiments, the subject may have not been previously treated with trastuzumab. In other embodiments, the subject may have not been previously treated with the oxaliplatin-based chemotherapy.

In other embodiments, the subject is a candidate to receive an oxaliplatin-based chemotherapy (e.g., a FOLFOX regimen, such as mFOLFOX7). In some examples, the subject can be previously treated or currently treated with an oxaliplatin-based chemotherapy. In some embodiments, subjects are receiving oxaliplatin at a dose of 85 mg/m² per 2-week cycle, or the subject is a candidate to receive an oxaliplatin-based chemotherapy as part of their standard-of-care treatment. In some embodiments, subjects with gastric or GEJ adenocarcinoma can also be receiving trastuzumab as part of their standard-of-care regimen if the oxaliplatin-based chemotherapy is part of a first-line therapy.

In some embodiments, subject are treated with an oxaliplatin-based chemotherapy, trastuzumab, and tucatinib given in a 14-day cycle. For example, subjects can be treated with oxaliplatin, leucovorin, and fluorouracil administered as an mFOLFOX7 regimen, trastuzumab, and tucatinib given in a 14-day cycle. In some embodiments, subjects may have received up to 28 days of oxaliplatin-based chemotherapy backbone (e.g., at a dose of 85 mg/m² per 2-week cycle) prior to receiving the combination therapy. In some embodiments, subjects will receive 7 days of an oxaliplatin-based chemotherapy (e.g., an oxaliplatin-based chemotherapy administered using an mFOLFOX7 regimen) and trastuzumab without tucatinib. The tucatinib can, in some embodiments, be provided as treatment on day 8 of a 14 day cycle. In some embodiments, the starting dose of tucatinib is 150 mg orally twice daily.

In some embodiments, the subject may have been previously treated with at least one other anticancer therapy. In some embodiments, the subject may have been previously treated with at least one anticancer therapy for HER2 positive cancer. Non-limiting examples of anticancer therapies include an anti-HER2 antibody, an anti-HER2 antibody-drug conjugate, trastuzumab, trastuzumab and a taxane, pertuzumab, ado-trastuzumab (T-DM1), and combinations thereof.

In some embodiments, the subject has been previously treated with one or more additional therapeutic agents for the cancer and did not respond to the treatment. In some embodiments, the subject has been previously treated with one or more additional therapeutic agents for the cancer and relapsed after the treatment. In some embodiments, the subject can be refractory or develop a brain metastasis during the previous anticancer therapy.

In some embodiments, the subject has been previously treated with one or more additional therapeutic agents for the cancer and experienced disease progression during the treatment. In some embodiments, the one or more additional therapeutic agents is an anti-HER2 antibody or anti-HER2 antibody-drug conjugate. In some embodiments, the one or more additional therapeutic agents is an anti-HER2 antibody. In some embodiments, the one or more additional therapeutic agents is anti-HER2 antibody-drug conjugate. In some embodiments, the subject has been previously treated with trastuzumab, trastuzumab and a taxane, pertuzumab and/or T-DM1. In some embodiments, the subject has been previously treated with trastuzumab. In some embodiments, the subject has been previously treated with pertuzumab. In some embodiments, the subject has been previously treated with T-DM1. In some embodiments, the subject has been previously treated with trastuzumab and pertuzumab. In some embodiments, the subject has been previously treated with trastuzumab and T-DM1. In some embodiments, the subject has been previously treated with pertuzumab and T-DM1. In some embodiments, the subject has been previously treated with trastuzumab, pertuzumab and T-DM1. In some embodiments, the subject has been treated with trastuzumab and a taxane. In some embodiments, the subject has been treated with trastuzumab and a taxane and has also been treated with pertuzumab.

In some embodiments, the one or more additional therapeutic agents are selected from the group consisting of chemotherapeutic agents such as doxorubicin and cyclophosphamide (e.g., ACTH regimen); a taxane (e.g., paclitaxel); docetaxel; docetaxel and carboplatin (e.g., TCH regimen); cisplatin; fluorouracil (e.g., 5-FU); epirubicin; anthracyclines (e.g., doxorubicin); cyclophosphamide; vinorelbine; gemcitabine; kinase inhibitors, such as lapatinib; neratinib; pyrotinib; afatinib; poziotinib; abemaciclib; and pazopanib; hormone therapy, including, for example, tamoxifen; toremifene; fulvestrant; aromatase inhibitors (e.g., anastrozole, exemestane, letrozole); and ovarian suppression (e.g., with goserelin or leuprolide); vaccines such as nelipepimut-S or E75 peptide combined with granulocyte macrophage-colony stimulating factor; and ETBX-021; combination therapies such as a chemotherapeutic agent and trastuzumab (and optionally pertuzumab); a taxane (e.g., paclitaxel) with trastuzumab; a taxane (e.g., paclitaxel) with trastuzumab and pertuzumab; cis-platin and fluoropyrimidine with trastuzumab; docetaxel and carboplatin with trastuzumab and pertuzumab; docetaxel and carboplatin with trastuzumab; docetaxel with trastuzumab and pertuzumab; docetaxel with trastuzumab; docetaxel and cyclophosphamide with trastuzumab; anthracycline and/or cyclophosphamide followed by paclitaxel with trastuzumab; pertuzumab with docetaxel; fluorouracil (e.g., 5-FU), epirubicin, and cyclophosphamide with trastuzumab and/or pertuzumab; vinorelbine or gemcitabine with trastuzumab; anthracycline, a taxane and trastuzumab; doxorubicin with trastuzumab; lapatinib with capecitabine; lapatinib with trastuzumab; endocrine therapy with lapatinib and/or trastuzumab; pazopanib with lapatinib; anti-HER2 agents (e.g., trastuzumab) with CDK4/6 inhibitors (e.g., abemaciclib or palbociclib) such as abemaciclib with trastuzumab; palbociclib with trastuzumab, pertuzumab, and an aromatase inhibitor; palbociclib, trastuzumab (and optionally letrozole); palbociclib and T-DM1; palbociclib with trastuzumab, pertuzumab and anastrozole; ribociclib with trastuzumab or T-DM1; palbociclib with tucatinib and letrozole; anti-HER2 agents (e.g., trastuzumab, pertuzumab, T-DM1) with immunotherapy (e.g., with pembrolizumab, atezolizumab or nivolumab); anti-HER2 agents (e.g., trastuzumab, pertuzumab, T-DM1) with PI3K/AKT/mTOR inhibitors, for example, everolimus with trastuzumab and paclitaxel; everolimus with trastuzumab and vinorelbine; alpelisib with LIM716 and trastuzumab; alpelisib and T-DM1; taselisib with anti-HER2 agents (e.g., trastuzumab, trastuzumab emtansine, pertuzumab (and optionally paclitaxel)); and copanlisib with trastuzumab.

In some embodiments, the subject has not been previously treated with another anticancer therapy for the cancer within the past 1 day, 2 days, 3 days, 4 days, 5 days, 6 days, 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not been previously treated with another therapeutic agent for the cancer within the past 12 months prior to being administered the therapeutically effective amount of tucatinib, or salt or solvate thereof. In some embodiments, the subject has not been previously treated with another therapeutic agent for the cancer. In some embodiments, the subject has not been previously treated with lapatinib, neratinib, afatinib, or capecitabine. In some embodiments, the subject has not been previously treated with lapatinib. In some embodiments, the subject has not been previously treated with neratinib. In some embodiments, the subject has not been previously treated with afatinib. In some embodiments, the subject has not been previously treated with capecitabine. In some embodiments, the subject has not been previously treated with an anti-HER2 antibody-drug conjugate (e.g., ado-trastuzumab emtansine).

In some embodiments, the HER2 status of a sample cell is determined. The determination can be made before treatment (i.e., administration of a combination of tucatinib, trastuzumab, and oxaliplatin-based chemotherapy) begins, during treatment, or after treatment has been completed. In some instances, determination of the HER2 status results in a decision to change therapy (e.g., adding, changing, or discontinuing the use of the combination of tucatinib, trastuzumab, and oxaliplatin-based chemotherapy, discontinuing therapy altogether, or switching from another treatment method to a method of the present disclosure).

In some embodiments, the sample cell is determined to be overexpressing or not overexpressing HER2. In particular embodiments, the cell is determined to be HER2 3+, HER2 2+, HER2 1+, or HER2 0 (i.e., HER is not overexpressed).

In some embodiments, the sample cell is a cancer cell. In some instances, the sample cell is obtained from a subject who has cancer. The sample cell can be obtained as a biopsy specimen, by surgical resection, or as a fine needle aspirate (FNA). In some embodiments, the sample cell is a circulating tumor cell (CTC).

HER2 expression can be compared to a reference cell. In some embodiments, the reference cell is a non-cancer cell obtained from the same subject as the sample cell. In other embodiments, the reference cell is a non-cancer cell obtained from a different subject or a population of subjects. In some embodiments, measuring expression of HER2 comprises, for example, determining HER2 gene copy number or amplification, nucleic acid sequencing (e.g., sequencing of genomic DNA or cDNA), measuring mRNA expression, measuring protein abundance, or a combination thereof. HER2 testing methods include immunohistochemistry (IHC), in situ hybridization, fluorescence in situ hybridization (FISH), chromogenic in situ hybridization (CISH), ELISAs, and RNA quantification (e.g., of HER2 expression) using techniques such as RT-PCR and microarray analysis.

In some embodiments, the sample cell is determined to be HER2 positive when HER2 is expressed at a higher level in the sample cell compared to a reference cell. In some embodiments, the cell is determined to be HER2 positive when HER2 is overexpressed at least about 1.5-fold (e.g., about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more) compared to a reference cell. In particular embodiments, the cell is determined to be HER2 positive when HER2 is overexpressed at least about 1.5-fold compared to the reference cell.

In some embodiments, the sample cell is determined to be HER2 positive when the FISH or CISH signal ratio is greater than 2. In other embodiments, the sample cell is determined to be HER2 positive when the HER2 gene copy number is greater than 6.

In one aspect, provided herein are methods for treating or ameliorating a HER2 positive cancer in a subject in need thereof, the method comprising administering to the subject a therapeutically effective amount of a combination therapy comprising tucatinib, trastuzumab and an oxaliplatin-based chemotherapy.

In one aspect provided herein are methods for treating a HER2 positive cancer in a subject that has exhibited an adverse event after starting treatment with a combination therapy comprising tucatinib, trastuzumab, and an oxaliplatin-based chemotherapy at an initial dosage level, comprising administering to the subject at least one of the combination therapy at a reduced dosage level. For example, one, two, or all of the components included in the combination therapy can be reduced. In such embodiments, an individual component (e.g., one of tucatinib, trastuzumab, or the oxaliplatin-based chemotherapy) of the combination therapy can be reduced following an adverse reaction experienced by the subject while the other components of the compound therapy remain at their initial dosage levels. In another such embodiment, two of the components (e.g., two of tucatinib, trastuzumab, or the oxaliplatin-based chemotherapy) of the combination therapy can be reduced following an adverse reaction experienced by the subject while the remaining component of the compound therapy remains at its initial dosage level. In another such embodiment, all of the components (e.g., tucatinib, trastuzumab, and the oxaliplatin-based chemotherapy) of the combination therapy can be reduced following an adverse reaction experienced by the subject and none of the components that comprise the compound therapy remain at the initial dosage level. For example, the methods can include methods for treating the HER2 positive cancer in a subject that has exhibited an adverse event after starting treatment with at least one of the combination therapy comprising tucatinib, trastuzumab, and oxaliplatin, leucovorin, and fluorouracil administered as an mFOLFOX7 regimen.

In some embodiments of any of the methods described herein, the method can further comprise treating a HER2 positive cancer in a subject in need thereof comprising administering to the subject a therapeutically effective amount of a combination therapy comprising tucatinib, trastuzumab, and an oxaliplatin-based chemotherapy, and administering an effective amount of an anti-diarrheal agent.

In some embodiments, the anti-diarrheal agent is administered prophylactically. In some embodiments of any of the methods described herein, the method can include reducing the severity or incidents of diarrhea, or preventing diarrhea in a subject having a HER2 positive cancer and being treated with an effective amount of a combination therapy comprising tucatinib, trastuzumab, and an oxaliplatin-based chemotherapy, the method comprising administering an effective amount of an anti-diarrheal agent prophylactically.

In another embodiment of any of the methods described herein, the method can include a method of reducing the likelihood of a subject developing diarrhea, wherein the subject has a HER2 positive cancer and is being treated with an effective amount of a combination therapy comprising tucatinib, trastuzumab, and an oxaliplatin-based chemotherapy the method comprising administering an effective amount of an anti-diarrheal agent prophylactically.

In some embodiments, the combination therapy and the anti-diarrheal agent are administered sequentially. In some embodiments, the combination therapy and the anti-diarrheal agent are administered concurrently. In some embodiments, the anti-diarrheal agent is administered prior to administration of the combination therapy. For example, one hour before, two hours before, four hours before, six hours before, twelve hours before, one day before, two days before, three days before, four days before, five days before, or one week before. In some cases, the subject is exhibiting symptoms of diarrhea prior to administration of the anti-diarrheal agent. In other cases, the subject is not exhibiting symptoms of diarrhea prior to administration of the anti-diarrheal agent.

Non-limiting examples of anti-diarrheal agents include loperamide, budesonide (e.g., in combination with loperamide), prophylactic antibiotics (e.g., doxycycline), probiotics, electrolyte replacement solutions, colestipol, colestipol in combination with loperamide, octreotide, crofelemer, TJ14, Bacillus Cereus, calcium aluminosilicate, sulfasalazine, cefpodoxime, elsiglutide, glutamine, codeine, diphenoxylate, atropine, bismuth subsalicylate, diphenoxylate, atropine, attapulgite, activated charcoal, bentonite, saccharomyces boulardii lyo, rifaximin, neomycin, alosetron, octreotide, crofelemer, opium, cholestyramine, and colesevelam.

In some embodiments, the combination therapy and the anti-diarrheal agent are administered sequentially. In some embodiments, the combination therapy and the anti-diarrheal agent are administered concurrently. In some embodiments, the anti-diarrheal agent is administered prior to administration of the combination therapy. For example, one hour before, two hours before, four hours before, six hours before, twelve hours before, one day before, two days before, three days before, four days before, five days before, or one week before. In some cases, the subject is exhibiting symptoms of diarrhea prior to administration of the antiemetic agent. In other cases, the subject is not exhibiting symptoms of diarrhea prior to administration of the antiemetic agent.

### B. Tucatinib Dose and Administration

In some embodiments, a dose of tucatinib is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, a dose of tucatinib is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In particular embodiments, a dose of tucatinib is between about 1 mg and 50 mg per kg of the subject's body weight (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per kg of the subject's body weight). In some instances, a dose of tucatinib is about 50 mg per kg of the subject's body weight.

In some embodiments, a dose of tucatinib comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of tucatinib. In other embodiments, a dose of tucatinib comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of tucatinib. In particular embodiments, a dose of tucatinib is about 300 mg (e.g., when administered twice per day). In certain of these embodiments, a dose of tucatinib is 300 mg (e.g., 6×50 mg tablets; or 2×150 mg tablets), administered twice per day.

In some embodiments, a dose of tucatinib, or salt or solvate thereof, contains a therapeutically effective amount of tucatinib, or salt or solvate thereof. In other embodiments, a dose of tucatinib, or salt or solvate thereof, contains less than a therapeutically effective amount of tucatinib, or salt or solvate thereof, (e.g., when multiple doses are given in order to achieve the desired clinical or therapeutic effect).

Tucatinib, or salt or solvate thereof, can be administered by any suitable route and mode. Suitable routes of administering combination therapies of the present disclosure are well known in the art and may be selected by those of ordinary skill in the art. In one embodiment, tucatinib administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of tucatinib is intravenous injection or infusion. In some embodiments, the route of administration of tucatinib is intravenous infusion. In some embodiments, the route of administration of tucatinib is intravenous injection or infusion. In some embodiments, the tucatinib is intravenous infusion. In some embodiments, the route of administration of tucatinib is oral.

In one embodiment of the methods or uses or product for uses provided herein, tucatinib is administered to the subject at a dose of about 100 mg to about 1000 mg (e.g., about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, or about 1000 mg).

In one embodiment of the methods or uses or product for uses provided herein, tucatinib is administered to the subject daily, twice daily, three times daily or four times daily. In some embodiments, tucatinib is administered to the subject every other day, once about every week or once about every three weeks. In some embodiments, tucatinib is administered to the subject once per day. In some embodiments, tucatinib is administered to the subject twice per day. In some embodiments, tucatinib is administered to the subject at a dose of about 100 mg twice per day to about 500 mg twice per day, (e.g., about 100 mg twice per day, about 200 mg twice per day, about 300 mg twice per day, about 400 mg twice per day, or about 500 mg twice per day). In some embodiments, tucatinib is administered to the subject at a dose of 300 mg twice per day. In some embodiments, tucatinib is administered to the subject at a dose of about 100 mg once per day to about 1000 mg once per day (e.g., 100 mg once per day, 200 mg once per day, 300 mg once per day, 400 mg once per day, 500 mg once per day, 600 mg once per day, 700 mg once per day, 800 mg once per day, 900 mg once per day, or 1000 meg once per day). In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally.

### C. Combination Therapy

Provided herein are methods of treatment comprising administering to the subject a combination therapy comprising tucatinib, trastuzumab, and an oxaliplatin-based chemotherapy. In some embodiments, the combination therapy consists essentially of tucatinib, trastuzumab, and an oxaliplatin-based chemotherapy.

In some embodiments, the tucatinib, trastuzumab, and an oxaliplatin-based chemotherapy are administered to the subject on a treatment cycle. In some embodiments, the tucatinib, trastuzumab, and an oxaliplatin-based chemotherapy are administered to a subject on a 14 day treatment cycle. In some embodiments, the subject will be treated with trastuzumab, tucatinib and an oxaliplatin-based chemotherapy that is administered as a regimen selected from the group consisting of FOLFOX4, mFOLFOX4, FOLFOX6, mFOLFOX6, FOLFOX7, mFOLFOX7, FOLFOXIRI, bFOL, PVIFOX, IROX, FUOX, FuFOX, CapeOx, XELOX, and CAPOX given in 14-day cycles. For instance, the subject can be treated with tucatinib, trastuzumab, and oxaliplatin, leucovorin, and fluorouracil administered as an mFOLFOX7 regimen given in 14-day cycles. An eligible subject may have received up to 28 days of oxaliplatin-based chemotherapy backbone (at a dose of 85 mg/m² per 2-week cycle) prior to receiving the combination treatment. In some embodiments, the subject will receive 7 days of oxaliplatin, leucovorin, and fluorouracil administered as an mFOLFOX7 regimen and trastuzumab without tucatinib. The starting dose of tucatinib is 150 mg orally (PO) BID, and the first dose will be administered on Cycle 1 Day 8 and continuously thereafter.

In some embodiments, tucatinib is administered to the subject at a dose of about 200 mg twice per day. In some embodiments, tucatinib is administered to the subject at a dose of about 250 mg twice per day. In some embodiments, tucatinib is administered to the subject at a dose of about 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 14 day treatment cycle. In some embodiments, tucatinib is administered to the subject twice per day beginning on day 8 of a 14 day cycle. In some embodiments, the tucatinib is administered to the subject orally. In some embodiments, the trastuzumab is administered to the subject at a dose of about 6 mg/kg.

### D. Compositions of Tucatinib

In some embodiments, a pharmaceutical composition comprising tucatinib and a pharmaceutically acceptable carrier is provided herein, wherein the pharmaceutical composition comprises a solid dispersion of tucatinib.

The solid dispersions are generally prepared by dissolving the drug substance and the dispersion polymer in a suitable solvent to form a feed solution, and then the feed solution may be spray dried to form the solid dispersion (and remove the solvent). Spray drying is a known process. Spray drying is generally performed by dissolving tucatinib and the dispersion polymer in a suitable solvent to prepare a feed solution. The feed solution may be pumped through an atomizer into a drying chamber. The feed solution can be atomized by conventional means known in the art, such as a two-fluid sonicating nozzle, a pressure nozzle, a rotating nozzle and a two-fluid non-sonicating nozzle. Then, the solvent is removed in the drying chamber to form the solid dispersion. A typical drying chamber uses hot gases, such as forced air, nitrogen, nitrogen-enriched air, or argon to dry particles. The size of the drying chamber may be adjusted to achieve particle properties or throughput. Although the solid dispersion are preferably prepared by conventional spray drying techniques, other techniques known in the art may be used, such as melt extrusion, freeze drying, rotary evaporation, drum drying or other solvent removal processes.

In some embodiments, a process of preparing a solid dispersion is provided, comprising: (a) dissolving tucatinib and a dispersion polymer in a suitable solvent; and (b) evaporating the solvent to form the solid dispersion. In certain embodiments, the evaporation of the solvent in step (b) is performed by spray drying, melt extrusion, freeze drying, rotary evaporation, drum drying or other solvent removal processes.

In certain embodiments, the dispersion polymer is selected from PVP-VA, methylacrylic acid methyl methacrylate copolymer, HPMCP, CAP, HPMCAS and HPMC and mixtures thereof. In certain embodiments, the dispersion polymer is selected from PVP-VA, methylacrylic acid methyl methacrylate copolymer, HPMCP, CAP, HPMCAS and HPMC. In certain embodiments, the dispersion polymer is selected from PVP-VA, Eudragit® L100, HPMCP H-55, CAP, HPMCAS Grade M, HPMC and mixtures thereof. In certain embodiments, the dispersion polymer is selected from PVP-VA, Eudragit® L100, HPMCP H-55, CAP, HPMCAS Grade M and HPMC.

In certain embodiments, the dispersion polymer is selected from PVP-VA, methylacrylic acid methyl methacrylate copolymer, HPMCP, CAP and HPMCAS, and mixtures thereof. In certain embodiments, the dispersion polymer is selected from PVP-VA, methylacrylic acid methyl methacrylate copolymer, HPMCP, CAP and HPMCAS. In certain embodiments, the dispersion polymer is selected from PVP-VA, Eudragit® L100, HPMCP H-55, CAP and HPMCAS Grade M, and mixtures thereof. In certain embodiments, the dispersion polymer is selected from PVP-VA, Eudragit® L100, HPMCP H-55, CAP and HPMCAS Grade M.

In certain embodiments, the dispersion polymer is selected from PVP-VA, methylacrylic acid methyl methacrylate copolymer, HPMCP, CAP and HPMC, and mixtures thereof. In certain embodiments, the dispersion polymer is selected from PVP-VA, methylacrylic acid methyl methacrylate copolymer, HPMCP, CAP and HPMC. In certain embodiments, the dispersion polymer is selected from PVP-VA, Eudragit® L100, HPMCP H-55, CAP and HPMC, and mixtures thereof. In certain embodiments, the dispersion polymer is selected from PVP-VA, Eudragit® L100, HPMCP H-55, CAP and HPMC

In certain embodiments, the dispersion polymer is selected from PVP-VA, methylacrylic acid methyl methacrylate copolymer, HPMCP and CAP, and mixtures thereof. In certain embodiments, the dispersion polymer is selected from PVP-VA, methylacrylic acid methyl methacrylate copolymer, HPMCP and CAP. In certain embodiments, the dispersion polymer is selected from PVP-VA, Eudragit® L100, HPMCP H-55 and CAP, and mixtures thereof. In certain embodiments, the dispersion polymer is selected from PVP-VA, Eudragit® L100, HPMCP H-55 and CAP. In certain embodiments, the dispersion polymer is PVP-VA.

In certain embodiments, the dispersion polymer is methylacrylic acid methyl methacrylate copolymer. In certain embodiments, the dispersion polymer is Eudragit®. In certain embodiments, the dispersion polymer is Eudragit® L100.

In certain embodiments, the dispersion polymer is HPMCP. In certain embodiments, the dispersion polymer is HPMCP H-55.

In certain embodiments, the dispersion polymer is CAP.

In certain embodiments, the dispersion polymer is HPMCAS. In certain embodiments, the dispersion polymer is HPMCAS Grade M.

In certain embodiments, the dispersion polymer is preferably neutral or basic.

In certain embodiments, the dispersion polymer is selected from PVP-VA and HPMC. In certain embodiments, the dispersion polymer is HPMC.

Suitable solvents are a solvent or mixture of solvents in which both tucatinib and the dispersion polymer have adequate solubility (solubility greater than 1 mg/mL). A mixture of solvents may be used if each component of the solid dispersion (i.e., tucatinib and dispersion polymer) require different solvents to obtain the desired solubility. The solvent may be volatile with a boiling point of 150° C. or less. In addition, the solvent should have relatively low toxicity and be removed from the dispersion to a level that is acceptable to The International Committee on Harmonization (“ICH”) guidelines. Removal of solvent to this level may require a subsequent processing step, such as tray drying. Examples of suitable solvents include, but are not limited to, alcohols, such as methanol (“MeOH”), ethanol (“EtOH”), n-propanol, isopropanol (“IPA”) and butanol; ketones, such as acetone, methyl ethyl ketone (“MEK”) and methyl isobutyl ketone; esters, such as ethyl acetate (“EA”) and propyl acetate; and various other solvents, such as tetrahydrofuran (“THF”), acetonitrile (“ACN”), methylene chloride, toluene and 1,1,1-trichloroethane. Lower volatility solvents, such as dimethyl acetate or dimethylsulfoxide (“DMSO”), may be used. Mixtures of solvents with water may also be used, so long as the polymer and tucatinib are sufficiently soluble to make the spray drying process practicable. Generally, due to the hydrophobic nature of low solubility drugs, non-aqueous solvents may be used, meaning the solvent comprises less than about 10 weight % water.

In certain embodiments, the suitable solvent is selected from MeOH and THF, and mixtures thereof. In certain embodiments, the suitable solvent is MeOH:THF solvent system of about 1:3. In certain embodiments, the suitable solvent is a 1:3 MeOH:THF solvent system.

In certain embodiments, the suitable solvent is selected from MeOH, THF and water, and mixtures thereof. In certain embodiments, the suitable solvent is selected from MeOH, THF and water. In certain embodiments, the suitable solvent is a THF:MeOH:water solvent system of about 80:10:10. In certain embodiments, the suitable solvent is a 80:10:10 THF:MeOH:water solvent system. In certain embodiments, the suitable solvent is a THF:MeOH:water solvent system of about 82:8:10. In certain embodiments, the suitable solvent is a 82:8:10 THF:MeOH:water solvent system. In certain embodiments, the suitable solvent is a THF:MeOH:water solvent system of about 82.2:8.2:9.6. In certain embodiments, the suitable solvent is a 82.2:8.2:9.6 THF:MeOH:water solvent system.

In certain embodiments, the amount of tucatinib in the solid dispersion ranges from about 0.1% to about 70% by weight relative to the dispersion polymer. In certain embodiments, the amount of tucatinib in the solid dispersion ranges from 0.1% to 70% by weight relative to the dispersion polymer.

In certain embodiments, the amount of tucatinib in the solid dispersion ranges from about 1% to about 60% by weight relative to the dispersion polymer. In certain embodiments, the amount of tucatinib in the solid dispersion ranges from 1% to 60% by weight relative to the dispersion polymer.

In certain embodiments, the amount of tucatinib in the solid dispersion ranges from about 5% to about 60% by weight relative to the dispersion polymer. In certain embodiments, the amount of tucatinib in the solid dispersion ranges from 5% to 60% by weight relative to the dispersion polymer.

In certain embodiments, the amount of tucatinib in the solid dispersion ranges from about 55% to about 65% by weight relative to the dispersion polymer. In certain embodiments, the amount of tucatinib in the solid dispersion ranges from 55% to 65% by weight relative to the dispersion polymer. In certain embodiments, the amount of tucatinib in the solid dispersion is about 60% by weight relative to the dispersion polymer. In certain embodiments, the amount of tucatinib in the solid dispersion is 60% by weight relative to the dispersion polymer.

In certain embodiments, the amount of tucatinib in the solid dispersion ranges from about 25% to about 35% by weight relative to the dispersion polymer. In certain embodiments, the amount of tucatinib in the solid dispersion ranges from 25% to 35% by weight relative to the dispersion polymer. In certain embodiments, the amount of tucatinib in the solid dispersion is about 30% by weight relative to the dispersion polymer. In certain embodiments, the amount of tucatinib in the solid dispersion is 30% by weight relative to the dispersion polymer.

In certain embodiments, the amount of tucatinib in the solid dispersion ranges from about 45% to about 55% by weight relative to the dispersion polymer. In certain embodiments, the amount of tucatinib in the solid dispersion ranges from 45% to 55% by weight relative to the dispersion polymer. In certain embodiments, the amount of tucatinib in the solid dispersion is about 50% by weight relative to the dispersion polymer. In certain embodiments, the amount of tucatinib in the solid dispersion is 50% by weight relative to the dispersion polymer.

In certain embodiments, the solid dispersion is an amorphous solid dispersion.

Another embodiment provides a pharmaceutical composition comprising a solid dispersion of tucatinib and a dispersion polymer, and a carrier or excipient.

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

The pharmaceutical compositions may also include one or more additional components, such as buffers, dispersion agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug, i.e., a compound described herein or pharmaceutical composition thereof, or aid in the manufacturing of the pharmaceutical product, i.e., medicament (see Ansel; Gennaro; and Rowe above). The components of the pharmaceutical composition should be pharmaceutically acceptable.

Certain embodiments provide a pharmaceutical composition comprising: (a) about 1 to about 70 weight % of a solid dispersion of tucatinib; (b) about 0.1 to about 20 weight % of a disintegrant; (c) about 0.1 to about 25 weight % of an osmogen; (d) about 0.1 to about 10 weight % of a glidant; (e) about 0.1 to about 10 weight % of a lubricant; and (f) about 0.1 to about 25 weight % of a binder/diluent.

In certain embodiments, the pharmaceutical composition comprises: (a) 1 to 70 weight % of a solid dispersion of tucatinib; (b) 0.1 to 20 weight % of a disintegrant; (c) 0.1 to 25 weight % of an osmogen; (d) 0.1 to 10 weight % of a glidant; (e) 0.1 to 10 weight % of a lubricant; and (f) 0.1 to 25 weight % of a binder/diluent.

Certain embodiments provide a pharmaceutical composition comprising: (a) about 25 to about 60 weight % of a solid dispersion of tucatinib; (b) about 5 to about 15 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a binder/diluent.

In certain embodiments, the pharmaceutical composition comprises: (a) 25 to 60 weight % of a solid dispersion of tucatinib; (b) 5 to 15 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a binder/diluent.

Certain embodiments provide a pharmaceutical composition comprising: (a) about 40 to about 60 weight % of a solid dispersion of tucatinib; (b) about 5 to about 15 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a binder/diluent.

In certain embodiments, the pharmaceutical composition comprises: (a) 40 to 60 weight % of a solid dispersion of tucatinib; (b) 5 to 15 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a binder/diluent.

Certain embodiments provide a pharmaceutical composition comprising: (a) about 1 to about 70 weight % of a solid dispersion of tucatinib; (b) about 0.1 to about 20 weight % of a disintegrant; (c) about 0.1 to about 25 weight % of an osmogen; (d) about 0.1 to about 10 weight % of a glidant; (e) about 0.1 to about 10 weight % of a lubricant; and (f) about 0.1 to about 25 weight % of a filler.

In certain embodiments, the pharmaceutical composition comprises: (a) 1 to 70 weight % of a solid dispersion of tucatinib; (b) 0.1 to 20 weight % of a disintegrant; (c) 0.1 to 25 weight % of an osmogen; (d) 0.1 to 10 weight % of a glidant; (e) 0.1 to 10 weight % of a lubricant; and (f) 0.1 to 25 weight % of a filler.

Certain embodiments provide a pharmaceutical composition comprising: (a) about 25 to about 60 weight % of a solid dispersion of tucatinib; (b) about 1 to about 10 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a filler.

In certain embodiments, the pharmaceutical composition comprises: (a) 25 to 60 weight % of a solid dispersion of tucatinib; (b) 1 to 10 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a filler.

Certain embodiments provide a pharmaceutical composition comprising: (a) about 40 to about 60 weight % of a solid dispersion of tucatinib; (b) about 1 to about 10 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a filler.

In certain embodiments, the pharmaceutical composition comprises: (a) 40 to 60 weight % of a solid dispersion of tucatinib; (b) 1 to 10 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a filler.

In certain embodiments, the osmogen is selected from NaCl and KCl, and mixtures thereof.

In certain embodiments, the lubricant is magnesium stearate.

In certain embodiments, the glidant is colloidal silicon dioxide.

In certain embodiments, the binder/diluent is microcrystalline cellulose. In certain embodiments, the binder/diluent acts as both a binder and a diluent.

In certain embodiments, the binder is microcrystalline cellulose.

In certain embodiments, the diluent is microcrystalline cellulose.

In certain embodiments, the filler is lactose.

In certain embodiments, the disintegrant is selected from crospovidone and sodium bicarbonate (NaHCO₃), and mixtures thereof. In certain embodiments, the disintegrant is selected from crospovidone and sodium bicarbonate. In certain embodiments, the disintegrant is sodium bicarbonate. In certain embodiments, the disintegrant is crospovidone.

In certain embodiments, the composition contains sodium bicarbonate. tucatinib may slowly degrade, through hydrolysis or other means, to a carbamate impurity:

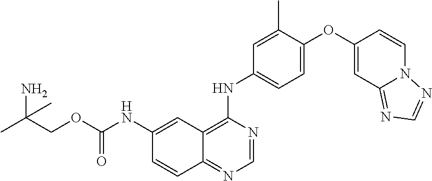 [SMILES: Cc1cc(Nc2ncnc3ccc(NC(=O)OCC(C)(C)N)cc23)ccc1Oc1ccn2ncnc2c1]

Sodium bicarbonate helps to slow the degradation to the carbamate impurity. Sodium bicarbonate also helps to provide consistent tablet disintegration when the tablets are exposed to different humidities.

Certain embodiments provide a pharmaceutical composition comprising: (a) tucatinib; and (b) sodium bicarbonate.

Certain embodiments provide a pharmaceutical composition comprising: (a) about 1 to about 70 weight % of a solid dispersion of tucatinib; and (b) about 0.1 to about 30 weight % sodium bicarbonate.

In certain embodiments, the pharmaceutical composition comprises: (a) 1 to 70 weight % of a solid dispersion of tucatinib; and (b) 0.1 to 30 weight % sodium bicarbonate.

Certain embodiments provide a pharmaceutical composition comprising: (a) about 1 to about 70 weight % of a solid dispersion of tucatinib; (b) about 0.1 to about 30 weight % sodium bicarbonate; and (c) the remaining weight is other pharmaceutically acceptable excipients and carriers.

In certain embodiments, the pharmaceutical composition comprises: (a) 1 to 70 weight % of a solid dispersion of tucatinib; (b) 0.1 to 30 weight % sodium bicarbonate; and (c) the remaining weight is other pharmaceutically acceptable excipients and carriers.

Certain embodiments provide a pharmaceutical composition comprising: (a) about 25 to about 60 weight % of a solid dispersion of tucatinib; and (b) about 1 to about 15 weight % of sodium bicarbonate.

In certain embodiments, the pharmaceutical composition comprises: (a) 25 to 60 weight % of a solid dispersion of tucatinib; and (b) 1 to 15 weight % of sodium bicarbonate.

Certain embodiments provide a pharmaceutical composition comprising: (a) about 25 to about 60 weight % of a solid dispersion of tucatinib; (b) about 1 to about 15 weight % of sodium bicarbonate; and (c) the remaining weight is other pharmaceutically acceptable excipients and carriers.

In certain embodiments, the pharmaceutical composition comprises: (a) 25 to 60 weight % of a solid dispersion of tucatinib; (b) 1 to 15 weight % of sodium bicarbonate; and (c) the remaining weight is other pharmaceutically acceptable excipients and carriers.

Certain embodiments provide a pharmaceutical composition comprising: (a) about 40 to about 60 weight % of a solid dispersion of tucatinib; and (b) about 1 to about 15 weight % of sodium bicarbonate.

In certain embodiments, the pharmaceutical composition comprises: (a) 40 to 60 weight % of a solid dispersion of tucatinib; and (b) 1 to 15 weight % of sodium bicarbonate.

Certain embodiments provide a pharmaceutical composition comprising: (a) about 40 to about 60 weight % of a solid dispersion of tucatinib; (b) about 1 to about 15 weight % of sodium bicarbonate; (c) the remaining weight is other pharmaceutically acceptable excipients and carriers.

In certain embodiments, the pharmaceutical composition comprises: (a) 40 to 60 weight % of a solid dispersion of tucatinib; (b) 1 to 15 weight % of sodium bicarbonate; (c) the remaining weight is other pharmaceutically acceptable excipients and carriers.

Certain embodiments provide a pharmaceutical composition comprising: (a) about 40 to about 60 weight % of a solid dispersion of tucatinib; (b) about 5 to about 15 weight % of a disintegrant which is selected from the group of crospovidone, sodium bicarbonate (NaHCO₃), and mixtures thereof; (c) about 15 to about 25 weight % of an osmogen which is selected from the group consisting of NaCl, KCl, and mixtures thereof; (d) about 0.1 to about 3 weight % of a glidant which is colloidal silicon dioxide; (e) about 0.1 to about 3 weight % of a lubricant which is magnesium stearate; and (f) about 10 to about 25 weight % of a binder/diluent which is microcrystalline cellulose.

In certain embodiments, the pharmaceutical composition comprises: (a) 40 to 60 weight % of a solid dispersion of tucatinib; (b) 5 to 15 weight % of a disintegrant which is selected from the group of crospovidone, sodium bicarbonate (NaHCO₃), and mixtures thereof; (c) 15 to 25 weight % of an osmogen which is selected from the group consisting of NaCl, KCl, and mixtures thereof; (d) 0.1 to 3 weight % of a glidant which is colloidal silicon dioxide; (e) 0.1 to 3 weight % of a lubricant which is magnesium stearate; and (f) 10 to 25 weight % of a binder/diluent which is microcrystalline cellulose.

Certain embodiments provide a pharmaceutical composition comprising: (a) about 40 to about 60 weight % of a solid dispersion of tucatinib; (b) about 1 to about 10 weight % of a disintegrant which is selected from the group of crospovidone, sodium bicarbonate (NaHCO₃), and mixtures thereof; (c) about 15 to about 25 weight % of an osmogen which is selected from the group consisting of NaCl, KCl, and mixtures thereof; (d) about 0.1 to about 3 weight % of a glidant which is colloidal silicon dioxide; (e) about 0.1 to about 3 weight % of a lubricant which is magnesium stearate; and (f) about 10 to about 25 weight % of a filler which is lactose.

In certain embodiments, the pharmaceutical composition comprises: (a) 40 to 60 weight % of a solid dispersion of tucatinib; (b) 1 to 10 weight % of a disintegrant which is selected from the group of crospovidone, sodium bicarbonate (NaHCO₃), and mixtures thereof; (c) 15 to 25 weight % of an osmogen which is selected from the group consisting of NaCl, KCl, and mixtures thereof; (d) 0.1 to 3 weight % of a glidant which is colloidal silicon dioxide; (e) 0.1 to 3 weight % of a lubricant which is magnesium stearate; and (f) 10 to 25 weight % of a filler which is lactose.

In certain embodiments, the pharmaceutical composition is selected from the group consisting of:

| Function | Ingredient | % of Blend |
|---|---|---|
| API | Solid dispersion of tucatinib | about 50 |
| Disintegrant | Crospovidone - Polyplasdone ® | about 6 |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| Binder/Diluent | Microcrystalline cellulose - Avicel ® | about 19.25 |
|---|---|---|
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |
| Function | Ingredient | % of Blend |
| API | Solid dispersion of tucatinib | about 50 |
| Disintegrant | Crospovidone - Polyplasdone ® | about 6 |
| Disintegrant | NaHCO₃ | about 3 |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline cellulose - Avicel ® | about 16.25 |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

| Function | Ingredient | % of Blend |
|---|---|---|
| API | Solid dispersion of tucatinib | about 50 |
| Disintegrant | Crospovidone - Polyplasdone ® | about 6 |
| Osmogen | NaCl | about 10.625 |
| Osmogen | KCl | about 10.625 |
| Filler | Lactose | about 21.25 |
| Glidant | Colloidal Silicon Dioxide | about 0.5 |
| Lubricant | Magnesium Stearate | aabout 0.25 |

Extragranular

| | | |
|---|---|---|
| Glidant | Colloidal Silicon Dioxide | about 0.5 |
| Lubricant | Magnesium Stearate | about 0.25 |

In certain embodiments, the pharmaceutical composition is selected from the group consisting of:

| Function | Ingredient | % of Blend |
|---|---|---|
| API | Solid dispersion of tucatinib | 50 |
| Disintegrant | Crospovidone - Polyplasdone ® | 6 |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| Binder/Diluent | Microcrystalline cellulose - Avicel ® | 19.25 |
|---|---|---|
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |
| Function | Ingredient | % of Blend |
| API | Solid dispersion of tucatinib | 50 |
| Disintegrant | Crospovidone - Polyplasdone ® | 6 |
| Disintegrant | NaHCO₃ | 3 |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline cellulose - Avicel ® | 16.25 |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

| Function | Ingredient | % of Blend |
|---|---|---|
| API | Solid dispersion of tucatinib | 50 |
| Disintegrant | Crospovidone - Polyplasdone ® | 6 |
| Osmogen | NaCl | 10.625 |
| Osmogen | KCl | 10.625 |
| Filler | Lactose | 21.25 |
| Glidant | Colloidal Silicon Dioxide | 0.5 |
| Lubricant | Magnesium Stearate | 0.25 |

Extragranular

| | | |
|---|---|---|
| Glidant | Colloidal Silicon Dioxide | 0.5 |
| Lubricant | Magnesium Stearate | 0.25 |

The pharmaceutical composition preferably contains a therapeutically effective amount of tucatinib. However, in some embodiments, each individual dose contains a portion of a therapeutically effective amount of tucatinib, such that multiple doses of the composition may be required (for example, two or more tablets are required for a therapeutically effective amount). Thus, in this application when it states that the pharmaceutical composition contains a therapeutically effective amount it means that the composition may be one dose (for example, one tablet) or multiple doses (for example, two tablets). In certain embodiments, the pharmaceutical composition contains between 1 and 500 mg of tucatinib.

In certain embodiments, the pharmaceutical composition contains between about 25 and about 400 mg of tucatinib. In certain embodiments, the pharmaceutical composition contains between 25 and 400 mg of tucatinib.

In certain embodiments, the pharmaceutical composition contains between about 25 and about 100 mg (e.g., about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg) of tucatinib. In certain embodiments, the pharmaceutical composition contains between 25 and 100 mg (e.g., 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg) of tucatinib. In certain embodiments, the pharmaceutical composition contains between about 25 and about 75 mg of tucatinib. In certain embodiments, the pharmaceutical composition contains between 25 and 75 mg of tucatinib. In certain embodiments, the pharmaceutical composition contains about 50 mg of tucatinib. In certain particular embodiments, the pharmaceutical composition contains 50 mg of tucatinib. In certain of the foregoing embodiments, the pharmaceutical composition is formulated as a tablet. As a non-limiting example, the pharmaceutical composition is formulated as a tablet and contains 50 mg of tucatinib.

In certain embodiments, the pharmaceutical composition contains between about 100 and about 300 mg (e.g., about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg) of tucatinib. In certain embodiments, the pharmaceutical composition contains between 100 and 300 mg (e.g., 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg) of tucatinib. In certain embodiments, the pharmaceutical composition contains between about 100 and about 200 mg of tucatinib. In certain embodiments, the pharmaceutical composition contains between 100 and 200 mg of tucatinib. In certain embodiments, the pharmaceutical composition contains between about 125 and about 175 mg of tucatinib. In certain embodiments, the pharmaceutical composition contains between 125 and 175 mg of tucatinib. In certain embodiments, the pharmaceutical composition contains about 150 mg of tucatinib. In certain particular embodiments, the pharmaceutical composition contains 150 mg of tucatinib. In certain of the foregoing embodiments, the pharmaceutical composition is formulated as a tablet. As a non-limiting example, the pharmaceutical composition is formulated as a tablet and contains 150 mg of tucatinib.

The pharmaceutical compositions described herein may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), ocular, vaginal, intraperitoneal, intrapulmonary and intranasal. If parenteral administration is desired, the compositions will be sterile and in a solution or suspension form suitable for injection or infusion.

The compounds may be administered in any convenient administrative form, e.g., tablets, powders, capsules, dispersions, suspensions, syrups, sprays, suppositories, gels, emulsions, patches, etc.

The pharmaceutical compositions described herein are typically administered orally. The pharmaceutical compositions described herein are typically administered as a tablet, caplet, hard or soft gelatin capsule, pill, granules or a suspension.

Additional examples of pharmaceutical compositions of tucatinib and methods of preparation thereof are described in U.S. Pat. No. 9,457,093, which is incorporated by reference herein in its entirety.

The pharmaceutical compositions described herein may comprise one or more polymorphs of tucatinib. Exemplary polymorphs of tucatinib and methods of preparation thereof are described in U.S. Pat. No. 9,168,254, which is incorporated by reference herein in its entirety.

In some embodiments, the pharmaceutical composition comprises amorphous tucatinib. In certain embodiments, tucatinib in the pharmaceutical composition is substantially amorphous (e.g., at least 80%, at least 85%, at least 90%, or at least 95% amorphous).

In some embodiments, the pharmaceutical composition comprises a crystalline polymorph of tucatinib. In certain embodiments, tucatinib in the pharmaceutical composition is substantially crystalline (e.g., at least 80%, at least 85%, at least 90%, or at least 95% crystalline).

In certain embodiments, the pharmaceutical composition comprises polymorph Form A of tucatinib as described in U.S. Pat. No. 9,168,254. In certain embodiments, tucatinib in the pharmaceutical composition is substantially in Form A (e.g., at least 80%, at least 85%, at least 90%, or at least 95% Form A).

In certain embodiments, the pharmaceutical composition comprises polymorph Form B of tucatinib as described in U.S. Pat. No. 9,168,254. In certain embodiments, tucatinib in the pharmaceutical composition is substantially in Form B (e.g., at least 80%, at least 85%, at least 90%, or at least 95% Form B).

In certain embodiments, the pharmaceutical composition comprises polymorph Form C of tucatinib as described in U.S. Pat. No. 9,168,254. In certain embodiments, tucatinib in the pharmaceutical composition is substantially in Form C (e.g., at least 80%, at least 85%, at least 90%, or at least 95% Form C).

In certain embodiments, the pharmaceutical composition comprises polymorph Form D of tucatinib as described in U.S. Pat. No. 9,168,254. In certain embodiments, tucatinib in the pharmaceutical composition is substantially in Form D (e.g., at least 80%, at least 85%, at least 90%, or at least 95% Form D).

In certain embodiments, the pharmaceutical composition comprises polymorph Form E of tucatinib as described in U.S. Pat. No. 9,168,254. In certain embodiments, tucatinib in the pharmaceutical composition is substantially in Form E (e.g., at least 80%, at least 85%, at least 90%, or at least 95% Form E).

In certain embodiments, the pharmaceutical composition comprises polymorph Form F of tucatinib as described in U.S. Pat. No. 9,168,254. In certain embodiments, tucatinib in the pharmaceutical composition is substantially in Form F (e.g., at least 80%, at least 85%, at least 90%, or at least 95% Form F).

In certain embodiments, the pharmaceutical composition comprises polymorph Form G of tucatinib as described in U.S. Pat. No. 9,168,254. In certain embodiments, tucatinib in the pharmaceutical composition is substantially in Form G (e.g., at least 80%, at least 85%, at least 90%, or at least 95% Form G).

In certain embodiments, the pharmaceutical composition comprises polymorph Form H of tucatinib as described in U.S. Pat. No. 9,168,254. In certain embodiments, tucatinib in the pharmaceutical composition is substantially in Form H (e.g., at least 80%, at least 85%, at least 90%, or at least 95% Form H).

In certain embodiments, the pharmaceutical composition comprises polymorph Form I of tucatinib as described in U.S. Pat. No. 9,168,254. In certain embodiments, tucatinib in the pharmaceutical composition is substantially in Form I (e.g., at least 80%, at least 85%, at least 90%, or at least 95% Form I).

In certain embodiments, the pharmaceutical composition comprises polymorph Form J of tucatinib as described in U.S. Pat. No. 9,168,254. In certain embodiments, tucatinib in the pharmaceutical composition is substantially in Form J (e.g., at least 80%, at least 85%, at least 90%, or at least 95% Form J).

In certain embodiments, the pharmaceutical composition comprises polymorph Form K of tucatinib as described in U.S. Pat. No. 9,168,254. In certain embodiments, tucatinib in the pharmaceutical composition is substantially in Form K (e.g., at least 80%, at least 85%, at least 90%, or at least 95% Form K).

In certain embodiments, the pharmaceutical composition comprises polymorph Form L of tucatinib as described in U.S. Pat. No. 9,168,254. In certain embodiments, tucatinib in the pharmaceutical composition is substantially in Form L (e.g., at least 80%, at least 85%, at least 90%, or at least 95% Form L).

In certain embodiments, the pharmaceutical composition comprises polymorph Form M of tucatinib as described in U.S. Pat. No. 9,168,254. In certain embodiments, tucatinib in the pharmaceutical composition is substantially in Form M (e.g., at least 80%, at least 85%, at least 90%, or at least 95% Form M).

In certain embodiments, the pharmaceutical composition comprises polymorph Form N of tucatinib as described in U.S. Pat. No. 9,168,254. In certain embodiments, tucatinib in the pharmaceutical composition is substantially in Form N (e.g., at least 80%, at least 85%, at least 90%, or at least 95% Form N).

In certain embodiments, the pharmaceutical composition comprises polymorph Form 0 of tucatinib as described in U.S. Pat. No. 9,168,254. In certain embodiments, tucatinib in the pharmaceutical composition is substantially in Form 0 (e.g., at least 80%, at least 85%, at least 90%, or at least 95% Form 0).

In certain embodiments, the pharmaceutical composition comprises polymorph Form P of tucatinib as described in U.S. Pat. No. 9,168,254. In certain embodiments, tucatinib in the pharmaceutical composition is substantially in Form P (e.g., at least 80%, at least 85%, at least 90%, or at least 95% Form P).

### E. Articles of Manufacture and Kits

In another aspect, the present disclosure provides an article of manufacture or kit for treating or ameliorating the effects of a HER2 positive cancer in a subject, the kit comprising tucatinib, trastuzumab, and an oxaliplatin-based chemotherapy.

The articles of manufacture or kits are suitable for treating or ameliorating the effects of HER2 positive and/or metastatic cancer in a subject. In some embodiments, the cancer is an advanced cancer. In some other embodiments, the cancer is a drug-resistant cancer. In some instances, the cancer is a multidrug-resistant cancer.

Materials and reagents to carry out the various methods of the present disclosure can be provided in articles of manufacture or kits to facilitate execution of the methods. As used herein, the term “kit” includes a combination of articles that facilitates a process, assay, analysis, or manipulation. In particular, kits of the present disclosure find utility in a wide range of applications including, for example, diagnostics, prognostics, therapy, and the like.

Articles of manufacture or kits can contain chemical reagents as well as other components. In addition, the articles of manufacture or kits of the present disclosure can include, without limitation, instructions to the user, apparatus and reagents for administering combinations of tucatinib, trastuzumab, and an oxaliplatin-based chemotherapy or pharmaceutical compositions thereof, sample tubes, holders, trays, racks, dishes, plates, solutions, buffers, or other chemical reagents. Articles of manufacture or kits of the present disclosure can also be packaged for convenient storage and safe shipping, for example, in a box having a lid.

### Exemplary Embodiments

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Phase 1b, Dose Escalation Study of Tucatinib in Combination with Trastuzumab and an Oxaliplatin-Based Chemotherapy for HER2+ Positive Cancers

Study Objectives

Primary

To determine the recommended dose of tucatinib when combined with trastuzumab and modified FOLFOX7 (mFOLFOX7; i.e., oxaliplatin, leucovorin, and fluorouracil administered as an mFOLFOX7 regimen) in subjects with human epidermal growth factor receptor 2 (HER2)+ gastrointestinal cancers.

Secondary

To evaluate the safety and tolerability of tucatinib in combination with trastuzumab and mFOLFOX7.

To evaluate the combination of tucatinib, trastuzumab, and mFOLFOX7 for potential nephrotoxicity.

To evaluate the pharmacokinetics (PK) of tucatinib.

To evaluate the PK of oxaliplatin in the presence and absence of tucatinib

Exploratory

To evaluate the antitumor activity of tucatinib given in combination with trastuzumab and mFOLFOX7.

To explore correlations between tissue and blood-based biomarkers and clinical outcomes

Study Population

This study will enroll subjects with unresectable or metastatic HER2+ gastric adenocarcinoma, gastroesophageal junction (GEJ) adenocarcinoma, esophageal adenocarcinoma, colorectal carcinoma (CRC), cholangiocarcinoma, and gallbladder carcinoma. Subjects should be receiving or should be candidates to receive an oxaliplatin-based regimen as part of their standard-of-care treatment prior to enrollment.

Subjects must meet all of the enrollment criteria to be eligible for this study. Eligibility criteria may not be waived by the investigator and are subject to review in the event of a good clinical practice audit and/or health regulatory authority inspection.

Number of Planned Subjects

Approximately 15-30 subjects will be enrolled and treated.

Study Design

This is a phase 1b, dose escalation study of tucatinib in combination with trastuzumab and an oxaliplatin-based chemotherapy (e.g., mFOLFOX7) for subjects with unresectable or metastatic HER2+ gastrointestinal cancers.

Subjects with cholangiocarcinoma, gallbladder carcinoma, or CRC should have evidence of HER2 overexpression or amplification in fresh or archival tumor tissue or HER2 amplification detected via blood-based NGS. Subjects with gastric, esophageal, or GEJ adenocarcinoma should have HER2+ disease based on an FDA approved HER2 test for gastroesophageal cancer.

Subjects should be receiving oxaliplatin at a dose of 85 mg/m² per 2-week cycle or should be candidates to receive an oxaliplatin-based chemotherapy as part of their standard-of-care treatment prior to enrollment. Subjects are eligible if they are receiving this regimen at any line of treatment for unresectable or metastatic disease (≥1 line of treatment). Subjects with gastric or GEJ adenocarcinoma should also be receiving trastuzumab as part of their standard-of-care regimen if given as part of first-line therapy. Concurrent treatment with anti-VEGF antibodies is prohibited while on trial. There is no upper limit for the number of prior lines of therapy received, and prior exposure to oxaliplatin is allowed, provided the subject had not experienced Grade ≥3 hypersensitivity due to oxaliplatin.

Enrolled subjects will be treated with mFOLFOX7, trastuzumab, and tucatinib given in 14-day cycles. Eligible subjects may have received up to 28 days of oxaliplatin-based chemotherapy backbone (at a dose of 85 mg/m² per 2-week cycle) prior to enrolling on study. After enrolling on study, subjects will receive 7 days of mFOLFOX7 and trastuzumab without tucatinib. The starting dose of tucatinib is 150 mg orally (PO) twice daily (BID), and the first dose will be administered on Cycle 1 Day 8 and continuously thereafter until the subject comes off study. Subjects will continue on therapy until disease progression, unacceptable toxicity, withdrawal of consent, death, or study closure.

Dose escalation will use a 3+3 design. Three subjects will initially be enrolled and treated at the 150 mg BID dose level. If no renal dose-limiting toxicities (DLTs) are observed at the 150 mg BID dose level, the Safety Monitoring Committee (SMC) may recommend escalating to the 300 mg BID dose level. A renal DLT is an increase in serum cystatin C (>1.5× baseline) occurring between the first dose of tucatinib and the end of Cycle 3. If no renal DLTs are observed in the 3 subjects treated at the 300 mg BID dose level, this will be declared the recommended dose of tucatinib with mFOLFOX7. If 1 renal DLT is observed at the initial 150 mg BID dose level, an additional 3 subjects (for a total of 6 evaluable subjects) will be treated at this dose level, and the SMC will evaluate dose escalation to 200 or 250 mg BID if no further renal DLTs are observed. If ≥2 renal DLTs are observed in the first 3 or 6 subjects treated at the initial 150 mg BID dose level, alternative dose levels and/or regimens <150 mg BID may be considered by the SMC.

Once the recommended dose level is declared by the SMC, additional subjects will be enrolled in order to bring the total number of subjects treated at the recommended dose level to 12 DLT-evaluable subjects (inclusive of the 3 to 6 subjects treated at that level during escalation). Additional efficacy cohorts and cohorts with alternate oxaliplatin-based chemotherapy may be added once the recommended dose has been established.

The primary endpoint will be the incidence of renal DLTs, and secondary safety endpoints include assessment of adverse events (AEs) and laboratory abnormalities, change in GFR (estimated using serum cystatin C) from baseline, vital signs, and frequency of dose modifications. PK assessments will include analysis of tucatinib concentrations in blood and analysis of oxaliplatin concentrations in blood and urine.

Investigational Product, Dose, and Mode of Administration

Tucatinib 150 mg will be administered PO BID from Cycle 1 Day 8 onwards. Dose escalation levels of tucatinib 200, 250, and 300 mg may be evaluated based on the incidence of renal DLTs observed.

Trastuzumab 6 mg/kg will be administered intravenously (IV) on Cycle 1 Day 1 and then will be administered at 4 mg/kg every 2 weeks starting on Cycle 2 Day 1.

Oxaliplatin 85 mg/m², leucovorin 200 mg/m², and fluorouracil 2400 mg/m² will be administered IV every 2 weeks starting from Cycle 1 Day 1 onwards. fluorouracil will be administered as a continuous infusion.

Duration of Treatment

Study treatment will continue until unacceptable toxicity, disease progression, withdrawal of consent, death, or study closure.

Efficacy Assessments

Disease response will be assessed by the investigator according to RECIST v1.1. Treatment decisions will be made based upon local assessment of radiologic scans. Radiographic disease assessments will evaluate all known sites of disease, preferably using high quality spiral contrast computed tomography (CT), and covering, at a minimum, the chest, abdomen, and pelvis. Positron emission tomography-CT scans (if high quality CT scan is included) and/or magnetic resonance imaging scans may also be used as appropriate, as well as additional imaging of any other known sites of disease (e.g., nuclear bone scan imaging for bone lesions). For each subject, the same imaging modality as used at screening/baseline should be used throughout the study. Disease assessments will be done at screening/baseline, and every 8 weeks for first 24 weeks then every 12 weeks, irrespective of dose interruptions.

Pharmacokinetic Assessments

Blood (oxaliplatin and tucatinib) and urine (oxaliplatin) samples for PK assessments will be collected at protocol-defined time points. Plasma concentrations of tucatinib will be analyzed using validated mass spectrometry methods. Concentrations of total platinum in plasma, free platinum in plasma ultrafiltrates, and platinum in urine will be determined using validated methods. Remaining PK samples will be archived and may be used for the analysis of administrated compounds or related species with exploratory, non-validated assays.

PK parameters will be calculated using standard non-compartmental methods. PK parameters to be estimated include, but are not limited to, area under the plasma concentration-time curve to the time of the last quantifiable concentration (AUClast), maximum observed concentration (Cmax), observed trough concentration in plasma (Ctrough; tucatinib only), and time of Cmax (Tmax).

Biomarker Assessments

HER2 status will be determined by historic NGS, IHC, FISH, or cell-free DNA-based NGS. Additional biomarker assessments may include HER2 status by IHC, in situ hybridization and tissue or blood-based NGS as well as an exploratory assessment of HER2 mutations or other mutations as potential biomarkers of response. This assessment may enable the correlation of additional biomarkers with treatment outcome and may ultimately guide or refine patient selection strategies to better match tucatinib regimens with tumor phenotype/genotype in the future.

Safety Assessments

Safety assessments will include surveillance and recording of AEs, physical examination findings, and laboratory tests. Assessment of cardiac ejection fraction will be performed by multi-gated acquisition (MUGA) scan or echocardiogram (ECHO).

Statistical Methods

The DLT-evaluable analysis set includes all subjects who meet one of the following criteria: (1) had a renal DLT or (2) had taken at least 75% of planned fluorouracil, oxaliplatin, and tucatinib doses and been followed for at least 2 cycles of study treatment (through the end of Cycle 3), inclusive of dose delays.

Safety and efficacy will be assessed using descriptive statistics, including the number of observations, mean, median, standard deviation, minimum and maximum for continuous variables, and the number and percentages (of non-missing) per category for categorical variables. Confirmed objective response rate per investigator is defined as the proportion of subjects with confirmed complete or partial response, per RECIST v1.1. The 2-sided 90% exact confidence interval using Clopper-Pearson method will be calculated for the response rates.

Objectives and Endpoints

This study will evaluate the safety and PK of tucatinib in combination with trastuzumab and mFOLFOX7 in subjects with unresectable or metastatic HER2+ gastrointestinal cancers. Specific objectives and corresponding endpoints for the study are summarized below

TABLE 1
Objectives and corresponding endpoints

| Primary Objective | Corresponding Primary Endpoint |
|---|---|
| To determine the recommended | Incidence of renal dose-limiting |
| dose of tucatinib when combined | toxicities (DLTs) |
| with trastuzumab and mFOLFOX7 | |
| in subjects with HER2+ | |
| gastrointestinal cancers | |
| Secondary Objectives | Corresponding Secondary Endpoint |
| To evaluate the safety and | Type, incidence, severity, |
| tolerability of tucatinib in | seriousness, and relatedness of |
| combination with trastuzumab | AEs and laboratory abnormalities |
| and mFOLFOX7 | Vital signs and other relevant |
| | safety variables |
| To evaluate the combination | Change in GFR (as estimated using |
| of tucatinib, trastuzumab, | serum cystatin C) from baseline |
| and mFOLFOX7 for potential | through 2 cycles of combination |
| nephrotoxicity | therapy |
| To evaluate the PK of | PK parameters of tucatinib |
| tucatinib | (including but not limited to AUCₗₐₛₜ, |
| | Cₘₐₓ, C_{trough}, and Tₘₐₓ) |
| To evaluate the PK of | PK parameters of oxaliplatin |
| oxaliplatin in the presence | (including but not limited to AUCₗₐₛₜ, |
| and absence of tucatinib | Cₘₐₓ, Tₘₐₓ) |
| Exploratory Objectives | Corresponding Exploratory Endpoints |
| To evaluate the antitumor | ORR (RECIST v1.1) per investigator |
| activity of tucatinib given | |
| in combination with trastuzumab | |
| and mFOLFOX7 | |
| To explore correlations | Potential biomarkers of response, |
| between tissue and blood-based | resistance, or toxicity may be |
| biomarkers and clinical outcomes | evaluated in tissue and blood |

Investigational Plan

Summary of Study Design

This is a phase 1b, dose escalation study of tucatinib in combination with trastuzumab and mFOLFOX7 for subjects with unresectable or metastatic HER2+ gastrointestinal cancers. A total of approximately 15 to 30 subjects will be enrolled across approximately 10 clinical sites. Subjects are eligible if they have one of the following cancers:
CRC
Gastric adenocarcinoma
Esophageal or GEJ adenocarcinoma
Cholangiocarcinoma
Gallbladder carcinoma

Subjects with cholangiocarcinoma, gallbladder carcinoma, or CRC should have evidence of HER2 overexpression or amplification in fresh or archival tumor tissue or HER2 amplification detected via blood-based next generation sequencing (NGS). Subjects with gastric, esophageal, or GEJ adenocarcinoma should have HER2+ disease based on an FDA approved HER2 test for gastroesophageal cancer.

Subjects should be receiving oxaliplatin at a dose of 85 mg/m² per 2-week cycle or should be candidates to receive an oxaliplatin-based chemotherapy as part of their standard-of-care treatment prior to enrollment. Subjects are eligible if they are receiving this regimen at any line of treatment for unresectable or metastatic disease (≥1 line of treatment). Subjects with gastric or GEJ adenocarcinoma should also be receiving trastuzumab as part of their standard-of-care regimen if given as part of first-line therapy. Concurrent treatment with anti-VEGF antibodies is prohibited while on trial. There is no upper limit for the number of prior lines of therapy received, and prior exposure to oxaliplatin is allowed, provided the subject had not experienced Grade ≥3 hypersensitivity due to oxaliplatin.

Enrolled subjects will be treated with modified FOLFOX7 (mFOLFOX7), trastuzumab, and tucatinib given in 14-day cycles. Eligible subjects may have received up to 28 days of oxaliplatin-based chemotherapy backbone (at a dose of 85 mg/m² per 2-week cycle) prior to enrolling on study. After enrolling on study, subjects will receive 7 days of mFOLFOX7 and trastuzumab without tucatinib. The starting dose of tucatinib is 150 mg orally (PO) BID, and the first dose will be administered on Cycle 1 Day 8 and continuously thereafter until the subject comes off study. Subjects should receive prophylaxis for chemotherapy induced nausea and vomiting as per the investigator's institutional standards. Subjects will continue on therapy until disease progression, unacceptable toxicity, withdrawal of consent, death, or study closure.

Dose escalation will use a 3+3 design described in Section 3.1.1. Three subjects will initially be enrolled and treated at the 150 mg BID dose level. If no renal dose-limiting toxicities (DLTs) are observed at the 150 mg BID dose level, the Safety Monitoring Committee (SMC) may recommend escalating to the 300 mg BID dose level. If no renal DLTs are observed in the 3 subjects treated at the 300 mg BID dose level, this will be declared the recommended dose of tucatinib with mFOLFOX7. If 1 renal DLT is observed at the initial 150 mg BID dose level, an additional 3 subjects (for a total of 6 evaluable subjects) will be treated at this dose level, and the SMC will evaluate dose escalation to 200 or 250 mg BID if no further renal DLTs are observed. If ≥2 renal DLTs are observed in the first 3 or 6 subjects treated at the initial 150 mg BID dose level, alternative dose levels and/or regimens <150 mg BID may be considered by the SMC.

Once the recommended dose level is declared by the SMC, additional subjects will be enrolled in order to bring the total number of subjects treated at the recommended dose level to 12 DLT-evaluable subjects (inclusive of the 3 to 6 subjects treated at that level during escalation). Additional efficacy cohorts and cohorts with alternate oxaliplatin-based chemotherapies may be added once the recommended dose has been established.

The primary endpoint will be the incidence of renal DLTs, and secondary safety endpoints include assessment of adverse events (AEs) and laboratory abnormalities, change in glomerular filtration rate (GFR) (estimated using serum cystatin C) from baseline, vital signs, and frequency of dose modifications. PK assessments will include analysis of tucatinib concentrations in blood and analysis of oxaliplatin concentrations in blood and urine.

Dose-Escalation Cohorts

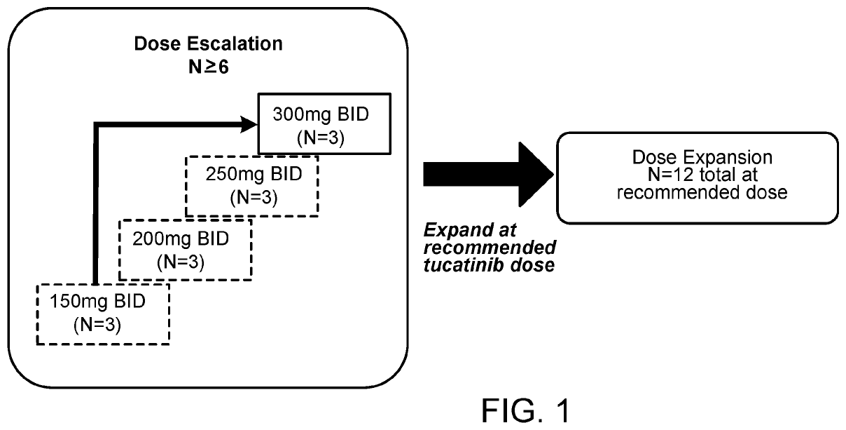

Three subjects will initially be enrolled and treated at the 150 mg BID dose level (FIG. 1). Once the 3 subjects are evaluable for renal DLT (Section 3.1.3), enrollment will be paused and the SMC will undertake a safety evaluation.

If no renal DLTs are observed at the 150 mg BID dose level, the SMC may recommend escalating to the 300 mg BID dose level, with 3 subjects being treated at this dose.

If no renal DLTs are observed in 3 subjects treated at the 300 mg BID dose level, this will be declared the recommended dose of tucatinib with mFOLFOX7.

If 1 renal DLT is observed in 3 subjects treated at the 300 mg BID dose level, an additional 3 subjects (for a total of 6 evaluable subjects) will be treated at this dose level. Once 6 subjects are evaluable for renal DLTs at 300 mg BID, enrollment will be paused, and the SMC will undertake a safety evaluation.

If ≤1 renal DLTs are observed in 6 evaluable subjects, this will be declared the recommended dose of tucatinib with mFOLFOX7.

If ≥2 renal DLTs are observed in the 6 subjects at the 300 mg dose level, dose levels of 250 and/or 200 mg may be explored, or an alternative dose level/schedule as recommended by the SMC.

If 1 renal DLT is observed at the 150 mg BID dose level, an additional 3 subjects (for a total of 6 evaluable subjects) will be treated at this dose level. Once 6 subjects are evaluable for renal DLTs, enrollment will be paused, and the SMC will undertake a safety evaluation. If ≤1 renal DLTs are observed in the 6 subjects treated at 150 mg BID, the SMC may recommend escalation to the 200 mg BID or 250 mg BID dose level. The SMC may also recommend an alternative dose level/schedule as warranted by the cumulative safety data.

If ≥2 renal DLTs are observed in the first 3 or 6 subjects treated at the 150 mg BID dose level, alternative dose levels and/or regimens <150 mg BID may be considered by the SMC.

At any intermediate dose level (200 or 250 mg BID), escalation will continue using the 3+3 design in increments of 50 mg until a dose of 300 mg BID is reached, or a recommended dose is declared at an alternate level. Enrollment will be paused after 3 and/or 6 subjects are enrolled at each level in order to evaluate renal DLTs and allow for an SMC meeting. If the dose escalation has proceeded from 150 mg to 250 mg, and ≥2 renal DLTs are observed in up to 6 evaluable subjects treated at this level, then the SMC may recommend dose de-escalation to the 200 mg dose level.

The recommended dose is the highest dose level where <1 renal DLTs are observed in 6 evaluable subjects. If at any point, the number of renal DLTs exceeds 33%, then that dose level will be declared above the recommended dose. Additional safety experiences in later cycles will also be considered when confirming the recommended dose.

In addition to renal DLTs, the SMC will take into consideration other AEs when making the determination to escalate, de-escalate, or declare the recommended dose level.

Expansion Cohort

Once the recommended dose of tucatinib in combination with trastuzumab and mFOLFOX7 is declared by the SMC, additional subjects will be enrolled in order to bring the total number of subjects treated at the recommended level to 12 DLT-evaluable subjects (inclusive of the 3 to 6 subjects treated at that level during escalation).

Renal Dose-Limiting Toxicity

A renal DLT is defined as an increase in serum cystatin C >1.5× baseline that is not related to pre-renal or post-renal etiologies (including disease progression, dehydration and intercurrent illness), and occurs during the period of treatment with tucatinib in combination with trastuzumab and mFOLFOX7 between the first dose of tucatinib and the end of Cycle 3.

Increases of serum cystatin C for which there is an alternative clinical explanation (e.g., clearly related to an intercurrent illness or disease progression) will not be considered renal DLTs. The relationship of an increase in serum cystatin C to study treatment will be determined by the investigator. In the event that it is not clear if the laboratory abnormality meets the criteria for a renal DLT, the study medical monitor should be contacted and the SMC consulted as needed.

Subjects will be considered DLT-evaluable if they have met one of the following criteria: (1) had a renal DLT or (2) had taken at least 75% of planned fluorouracil, oxaliplatin, and tucatinib doses and been followed for at least 2 cycles of study treatment (through the end of Cycle 3), inclusive of dose delays. Subjects that are not DLT-evaluable will be replaced.

The starting dose level of tucatinib will be 150 mg PO BID. This dose will be initiated on Cycle 1 Day 8, which will allow assessment of oxaliplatin PK in the absence of tucatinib and will be given continuously with each subsequent 14-day cycle of treatment until unacceptable toxicity, disease progression, withdrawal of consent, death, or study closure.

Study Population

Subjects must meet all of the enrollment criteria to be eligible for this study. Eligibility criteria may not be waived by the investigator and are subject to review in the event of a good clinical practice audit and/or health regulatory authority inspection.

Inclusion Criteria
1. Subjects must have unresectable or metastatic solid malignancy that is histologically or cytologically confirmed to be one of the tumor types listed below:
   CRC
   Gastric adenocarcinoma
   Esophageal and GEJ adenocarcinoma
   Cholangiocarcinoma
   Gallbladder carcinoma
2. Subjects must be receiving or must be candidates to receive an oxaliplatin-based chemotherapy as part of their standard-of-care treatment
   If subject is receiving oxaliplatin at the time of enrollment, the oxaliplatin dose in their current regimen must be 85 mg/m² per 2-week cycle.
3. HER2+ disease, as determined by laboratory testing based on one of the following:
   For colorectal, cholangiocarcinoma, and gallbladder carcinoma:
   HER2 amplification or overexpression from fresh or archival tumor tissue utilizing one of the following Clinical Laboratory Improvement Amendments (CLIA) certified tests:
      HER2 overexpression (3+ immunohistochemistry [IHC])
      HER2 (ERBB2) amplification by in situ hybridization assay (fluorescence in situ hybridization [FISH] or chromogenic in situ hybridization signal ratio ≥2.0 or gene copy number >6)
      HER2 (ERBB2) amplification by NGS assay
   HER2 amplification in a CLIA certified blood-based NGS assay
   Gastric, GEJ, and esophageal adenocarcinomas must use the following criteria:
   HER2+ overexpression (IHC3+) by an FDA approved assay, from a newly obtained biopsy or surgical specimen, evaluated following the package insert's interpretational manual for gastric adenocarcinoma. IHC2+ is eligible if the tumor is HER2 amplified by an FDA approved in situ hybridization assay
4. Measurable or non-measurable disease according to RECIST v1.1 as determined by the investigator.
5. Age 18 years or older.
6. An Eastern Cooperative Oncology Group (ECOG) Performance Status score of 0 or 1.
7. Life expectancy ≥3 months, in the opinion of the investigator
8. Adequate hepatic function, as defined by:
   Total bilirubin ≤1.5×ULN, except for subjects with known Gilbert's disease, who may enroll if the conjugated bilirubin is ≤1.5×ULN
   Transaminases (AST and ALT) ≤2.5×ULN (≤5×ULN if liver metastases are present)
9. Adequate baseline hematologic parameters as defined by:
   ANC≥1.5×10³/μL
   Platelet count ≥100×10³/μL; subjects with stable platelet count from 75-100×10³/μL may be included with approval from the medical monitor
   Hemoglobin ≥8 g/dL
   In subjects transfused before study entry, transfusion must be ≥14 days prior to start of therapy to establish adequate hematologic parameters independent from transfusion support
10. Estimated GFR:
   Dose Cohort #1: ≥90 mL/min/1.73 m² using the Modification of Diet in Renal Disease (MDRD) equation as applicable.
   Subjects with estimated GFR≥60 to <90 mL/min/1.73 m² may be enrolled in subsequent dose cohorts with medical monitor approval.
11. International normalized ratio (INR) and partial thromboplastin time (PTT)/activated partial thromboplastin time (aPTT)≤1.5×ULN, unless on medication known to alter INR and PTT/aPTT.
12. Left ventricular ejection fraction (LVEF)≥50% as assessed by echocardiogram (ECHO) or multigated acquisition (MUGA) scan documented within 4 weeks prior to first dose of study treatment
13. Subjects of childbearing potential, as defined in Section 4.3, under the following conditions:
   a. Must have a negative serum or urine pregnancy test (minimum sensitivity 25 mIU/mL or equivalent units of beta human chorionic gonadotropin [β-hCG]) result within 7 days prior to the first dose of study treatment. Subjects with false positive results and documented verification that the subject is not pregnant are eligible for participation.
   b. Must agree not to try to become pregnant during the study and for at least 7 months after the final dose of study drug
   c. Must agree not to breastfeed or donate ova, starting at time of informed consent and continuing through 7 months after the final dose of study drug
   d. If sexually active in a way that could lead to pregnancy, must consistently use 2 highly effective methods of birth control starting at the time of informed consent and continuing throughout the study and for at least 7 months after the final dose of study drug
14. Subjects who can father children, under the following conditions:
   a. Must agree not to donate sperm starting at time of informed consent and continuing throughout the study period and for at least 7 months after the final dose of study drug.
   b. If sexually active with a person of childbearing potential in a way that could lead to pregnancy, must consistently use 2 highly effective methods of birth control starting at time of informed consent and continuing throughout the study and for at least 7 months after the final dose of study drug.
   c. If sexually active with a person who is pregnant or breastfeeding, must consistently use one of 2 contraception options starting at time of informed consent and continuing throughout the study and for at least 7 months after the final dose of study drug.
15. The subject must provide written informed consent.
16. Subject must be willing and able to comply with study procedures, laboratory tests, and other requirements of the study

Exclusion Criteria
1. History of allergic reactions to oxaliplatin, fluorouracil, leucovorin, trastuzumab, or compounds chemically or biologically similar to tucatinib, except for Grade 1 or 2 infusion related reactions to oxaliplatin or trastuzumab that were successfully managed, or known allergy to any of the excipients in the study drugs
2. Current treatment regimen with an oxaliplatin dose >85 mg/m² per 2-week cycle or with an oxaliplatin dose 85 mg/m² per 2-week cycle that was initiated more than 28 days prior to enrollment
3. Major surgery within 28 days prior to enrollment
4. Subjects with known active central nervous system metastasis (irradiated or resected lesions are permitted, provided the lesions are fully treated and inactive, subject is asymptomatic, and no steroids have been administered for at least 30 days)
5. Any toxicity related to prior cancer therapies that has not resolved to ≤Grade 1, with the following exceptions:
   Alopecia;
   Clinically insignificant electrolyte abnormalities
6. Clinically significant cardiopulmonary disease such as:
   Ventricular arrhythmia requiring therapy 5. Any toxicity related to prior cancer therapies that has not resolved to ≤Grade 1, with the following exceptions:
   Ventricular arrhythmia requiring therapy
   Symptomatic hypertension or uncontrolled asymptomatic hypertension as determined by the investigator
   Any history of symptomatic CHF, left ventricular systolic dysfunction or decrease in ejection fraction
   Severe dyspnea at rest (Common Terminology Criteria for Adverse Events [CTCAE] Grade 3 or above) due to complications of advanced malignancy or hypoxia requiring supplementary oxygen therapy
   ≥Grade 2 QTc prolongation on screening electrocardiogram (ECG)
7. Known myocardial infarction or unstable angina within 6 months prior to first dose of study treatment
8. Known carrier of Hepatitis B or Hepatitis C or has other known chronic liver disease
9. Known to be positive for human immunodeficiency virus.
10. Subjects who are pregnant, breastfeeding, or planning to become pregnant from time of informed consent until 7 months following the last dose of study drug
11. Unable to swallow pills or has significant gastrointestinal disease which would preclude the adequate oral absorption of medications
14. Other medical, social, or psychosocial factors that, in the opinion of the investigator, could impact safety or compliance with study procedures
15. Evidence within 2 years of the start of study treatment of another malignancy that required systemic treatment
16. Any uncontrolled Grade 3 or higher (per National Cancer Institute [NCI] CTCAE v5.0) viral, bacterial, or fungal infection within 2 weeks prior to the first dose of study drug. Routine antimicrobial prophylaxis is permitted.

Treatments Administered

Subjects in the study will receive combination therapy of tucatinib with trastuzumab and mFOLFOX7 in 14-day cycles (Table 2). Subjects will receive trastuzumab and mFOLFOX7 starting on Cycle 1 Day 1, and the first dose of tucatinib will be administered on Cycle 1 Day 8. The starting dose of tucatinib is 150 mg PO BID.

TABLE 2
mFOLFOX7 and trastuzumab administered in 14-day cycles

| Drug | Dose/Route | | Administration | Days |
|---|---|---|---|---|
| Oxaliplatin | 85 | mg/m² IV | Administer over | Day 1 |
| | | | 2 h | |
| Leucovorina | 200 | mg/m² IV | Administer over | Day 1 |
| | | | 2 h concurrent | |
| | | | with oxaliplatin | |
| Fluorouracil | 2400 | mg/m² IV | Administer over | Day 1 |
| | | | 46 h | |
| Trastuzumab | 6 mg/kg IV (loading | | Administer over | Day 1 |
| | dose) | | 90 min | (Cycle |
| | | | | 1 only) |
| Trastuzumab | 4 | mg/kg IV | Administer over | Day 1 of |
| | | | 30 min | every |
| | | | | subsequent |
| | | | | cycle |

a Levoleucovorin can be substituted based on investigator preference in cases of supply shortages. Levoleucovorin is dosed at 1-half the usual dose of racemic d,l-leucovorin.
Study personnel should instruct subjects on tucatinib administration techniques and drug diary prior to Cycle 1 Day 8 and ensure that the subject understands these instructions before granting treatment independence.

Investigational Study Drug (Tucatinib)

Tucatinib drug product is supplied as both a coated yellow oval-shaped tablet in a 150 mg dosage strength and a coated yellow round convex tablet in a 50 mg dosage strength. The tablets are manufactured from a drug product intermediate amorphous dispersion of tucatinib in polyvinylpyrrolidone-vinyl acetate copolymer, which is then combined with the pharmaceutical excipients (microcrystalline cellulose, sodium chloride, potassium chloride, sodium bicarbonate, silicon dioxide, crospovidone, and magnesium stearate), and compressed into tablets.

Dose and Administration

The investigational study drug (tucatinib) will be administered PO BID. Subjects will be instructed by the pharmacist or investigator as to the specific number of tablets required for each dose. At each visit during study treatment, subjects will be supplied with the appropriate number of tablets for the number of doses to be taken prior to the next scheduled visit.

Subjects will be instructed to take tucatinib tablets twice each day (once in the morning, and once in the evening) approximately 8 to 12 hours between doses in the same calendar day. It is recommended that if a subject misses a scheduled dose of tucatinib and less than 6 hours have passed since the scheduled dosing time, the dose should be immediately taken. It is recommended that if more than 6 hours have passed since the scheduled dosing time, the subject should not take the missed dose but should wait and take the next regularly scheduled dose. Tablets may be taken with or without food. Tablets must be swallowed whole and may not be crushed, chewed or dissolved in liquid. On the day of dosing, the individual unit dose of the tucatinib tablet may be exposed to ambient temperature for up to 6 hours prior to dose.

Complete dosing instructions will be provided to the pharmacist prior to the initiation of the study. Complete dosing instructions will also be provided to study subjects and will include the minimum times between doses, dosing in relation to meals, and instructions for missed doses. Subjects will be instructed to take their morning dose of tucatinib in the clinic at the start of the oxaliplatin infusion on days when samples will be collected for PK analysis (Section 7.3). Subject compliance with investigational study drug dosing instructions will be assessed with the use of subject diaries and study drug accountability.

mFOLFOX7

Dose, Preparation, and Administration

For preparation and complete prescribing information for FOLFOX chemotherapy agents, please refer to the most current package inserts.

Administration of mFOLFOX7 will commence on Day 1 of each treatment cycle and will be administered every 2 weeks as described in Table 2. Oxaliplatin 85 mg/m² and leucovorin 200 mg/m² will be administered IV over 120 min. After completion of the leucovorin and oxaliplatin infusions, fluorouracil 2400 mg/m² will be administered IV over 46 hours. If there is a change in body weight of >10% from baseline, doses should be recalculated.

mFOLFOX7 Dose Modifications

Doses of fluorouracil, oxaliplatin, and leucovorin may be adjusted depending on an individual subject's tolerance. Table 4, Table 5, and Table 6 indicate recommended dose levels and modification guidelines for oxaliplatin and fluorouracil for non-neurological and neurological toxicity.

TABLE 3
mFOLFOX7 dose reduction levels (non-neurological toxicity)

| | Starting Dose | Dose Level-1 | Dose Level-2 |
|---|---|---|---|
| Oxaliplatin | 85 mg/m² | 65 mg/m² | 50 mg/m² |
| Fluorouracil | 2400 mg/m² | 2000 mg/m² | 1600 mg/m² |
| infusion | | | |

TABLE 4
mFOLFOX7 dose modifications for adverse events

| | Subsequent Cycles | |
|---|---|---|
| Toxicity CTCAE | Based on Interval | Prior to Each |
| Grade (v5.0) | Toxicity | Treatment Cycle |
| Neutropenia (ANC) | Maintain dose level | If ANC <1.5 × 10⁹/L at |
| Grade 1 | Maintain dose level | start of cycle, hold and |
| Grade 2 | Decrease both 5-FU | check weekly then treat |
| Grade 3 | and oxaliplatin 1 | based on interval toxicity |
| Grade 4 | dose level | If ANC <1.5 × 10⁹/L |
| | Decrease both 5-FU | after 4 weeks, discontinue |
| | and oxaliplatin 1 | therapy |
| | dose level | |
| Thrombocytopenia | Maintain dose level | If PLT <75 × 10⁹/L at |
| Grade 1 | Maintain dose level | start of cycle, hold and |
| Grade 2 | Decrease both 5-FU | check weekly then treat |
| Grade 3 | and oxaliplatin 1 | based on interval toxicity |
| Grade 4 | dose level | If PLT <75 × 10⁹/L |
| | Decrease both 5-FU | after 4 weeks, discontinue |
| | and oxaliplatin 1 | therapy |
| | dose level | |
| Leukopenia | Maintain dose level | If WBC <3.0 × 10⁹/L at |
| Grade 1 | Maintain dose level | start of cycle, hold and |
| Grade 2 | Decrease both 5-FU | check weekly then treat |
| Grade 3 | and oxaliplatin 1 | based on interval toxicity |
| Grade 4 | dose level | If WBC <3.0 × 10⁹/L |
| | Decrease both 5-FU | after 4 weeks, discontinue |
| | and oxaliplatin 1 | therapy |
| | dose level | |

| | |
|---|---|
| Febrile | Decrease both 5-FU and |
| neutropenia | oxaliplatin 1 dose level |

| | | |
|---|---|---|
| Diarrhea | Maintain dose level | If Grade ≥2 diarrhea at |
| Grade 1 | Maintain dose level | start of cycle, hold and |
| Grade 2 | Decrease both 5-FU | check weekly then treat |
| Grade 3 | and oxaliplatin 1 | based on interval toxicity |
| Grade 4 | dose level | |
| | Decrease both 5-FU | |
| | and oxaliplatin 1 | |
| | dose level | |

| | |
|---|---|
| Other | Dose modifications for other nonhematologic AEs related |
| nonhematologic | to therapy that are observed at the start of subsequent |
| toxicities | course and at the time of retreatment are based on CTCAE |
| | v5.0 and follow the same criteria as diarrhea above. |

Leucovorin dose modifications may be made at the investigator's discretion and per institutional standards.

TABLE 5
Dose modification for oxaliplatin-associated neurotoxicity

| | Duration of Neurotoxicity | | |
|---|---|---|---|
| | | | Persistent |
| | | | (Not Resolved |
| | 1-7 Days | >7 Days | Between Cycles) |
| Paresthesias/dysesthesias | No Change | No Change | No Change |
| of short duration that | | | |
| resolve and do not | | | |
| interfere with function | | | |
| (Grade 1) | | | |
| Paresthesias/dysesthesias | No Change | No Change | Decrease to |
| interfering with instrumental | | | 65 mg/m² |
| activities of daily living | | | |
| (Grade 2) | | | |
| Paresthesias/dysesthesias | 1st time: Decrease to | STOP | STOP |
| with pain or with functional | 65 mg/m² | | |
| impairment that interfere | 2nd time: 40 mg/m² | | |
| with self-care ADL | | | |
| (Grade 3) | | | |
| Persistent paresthesias/ | STOP | STOP | STOP |
| dysesthesias that are | | | |
| life-threatening | | | |
| (Grade 4) | | | |
| Pharyngo-laryngeal | No Change | Increase duration of | Increase duration of |
| dysesthesias | | infusion to 6 hours | infusion to 6 hours |

a AE severity graded using the NCI CTCAE v5.0.

A new cycle of mFOLFOX7 will be repeated every 2 weeks but may not be administered if the ANC<1.5×109/L, platelets <75.0×109/L, white blood cells <3.0×109/L, or diarrhea have not recovered to ≤Grade 1. Up to a 4-week delay is allowed in the initiation of a new cycle of treatment for resolution of toxicities. A treatment delay of 1 component of the mFOLFOX7 regimen (i.e., fluorouracil/leucovorin, or oxaliplatin) results in a similar delay of the other component to allow both therapies to be given together on Day 1 of each 2-week cycle.

In the event that oxaliplatin administration is discontinued for any reason prior to disease progression, fluorouracil/leucovorin, trastuzumab, and tucatinib therapy may be continued until disease progression. In the event that mFOLFOX7 chemotherapy administration is discontinued for any reason prior to disease progression, trastuzumab and tucatinib therapy may be continued until disease progression.

If mFOLFOX7 chemotherapy interruptions are ≤6 weeks from the previous cycle and the subject has recovered from toxicities as specified above, and the subject's disease has not progressed, mFOLFOX7 should be restarted at doses according to the tables above. If the mFOLFOX7 chemotherapy interruption is >6 weeks, but the subject has recovered from toxicity and the subject's disease has not progressed, then the subject may continue mFOLFOX7 therapy after consultation with and approval by the sponsor's medical monitor.

Trastuzumab

Dose, Preparation, and Administration

Trastuzumab will be given as a loading dose of 6 mg/kg IV (over 90 min) followed by 4 mg/kg (over 30 min) every 2 weeks (Table 2). Trastuzumab infusion rates will be per institutional guidelines. If dosing of trastuzumab has been held for >4 weeks, the IV loading dose of 6 mg/kg should be given per approved dosing instructions.

Single-dose vial (150 mg/vial) as a lyophilized sterile powder for reconstitution is commercially available and should be prepared and administered per instructions in the trastuzumab (Herceptin®) package insert. Trastuzumab will be administered IV under the direction of the investigator (subcutaneous administration is not allowed on study).

Trastuzumab should be stored according to the package insert.

Trastuzumab Dose Modifications

In the event of Grade ≥3 trastuzumab-related AEs other than infusion-related reactions (IRRs) (Section 5.5.4), hold trastuzumab until the AE resolved to Grade ≤1 or pretreatment levels and initiate or intensify applicable medical therapy, as appropriate. Resume trastuzumab at the same dose; the trastuzumab dose may not be reduced. If dosing of trastuzumab is held for >4 weeks and the medical monitor has agreed to restart trastuzumab, the IV loading dose of 6 mg/kg should be given per approved dosing instructions. Trastuzumab should be discontinued if a delay greater than 6 weeks is required due to treatment-related toxicity, unless a longer delay is approved by the study medical monitor.

Dose Modifications for Left Ventricular Dysfunction

Trastuzumab can cause left ventricular cardiac dysfunction, arrhythmias, hypertension, disabling cardiac failure, cardiomyopathy, and cardiac death. Trastuzumab can also cause asymptomatic decline in LVEF.

Trastuzumab dose modification guidelines for left ventricular dysfunction, regardless of relationship to study drug, are provided in Table 7.

TABLE 6
Dose modifications guidelines for left ventricular dysfunction

| | | LVEF 40% | LVEF 40% | |
|---|---|---|---|---|
| | | to ≤45% | to ≤45% | |
| Symptomatic | | and decrease | and decrease | |
| congestive | | is ≥10% points | is <10% points | |
| heart failure | LVEF <40% | from baseline | from baseline | LVEF >45% |
| Discontinue | do not administer | Do not administer | continue treatment | continue treatment |
| trastuzumab | trastuzumab. | trastuzumab. | with trastuzumab. | with trastuzumab |
| | repeat lvef | repeat lvef | repeat LVEF | |
| | assessment | assessment | assessment | |
| | within 4 weeks. | within 4 weeks. | within 4 weeks | |
| | if LVEF <40% is | If the LVEF has | | |
| | confirmed | not recovered to | | |
| | | within 10% points | | |
| | | from baseline, | | |
| | | discontinue | | |
| | | trastuzumab. | | |

Infusion-Related Reactions

Symptoms of IRR occurring after administration of trastuzumab, oxaliplatin, leucovorin, or fluorouracil include fever and chills, and on occasion included nausea, vomiting, pain (in some cases at tumor sites), headache, dizziness, dyspnea, hypotension, rash, and asthenia. In severe cases, symptoms have included bronchospasm, anaphylaxis, angioedema, hypoxia, and severe hypotension, usually reported during or immediately following the initial infusion. However, the onset and clinical course are variable, including progressive worsening, initial improvement followed by clinical deterioration, or delayed post-infusion events with rapid clinical deterioration. For fatal events, death occurred within hours to days following a serious infusion reaction. Interrupt infusion in all subjects experiencing dyspnea or clinically significant hypotension, and administer supportive therapy (which may include epinephrine, corticosteroids, diphenhydramine, bronchodilators, and oxygen). Subjects should be evaluated and carefully monitored until complete resolution of signs and symptoms. In subsequent infusions, premedicate subjects with antihistamines and/or corticosteroids.

Discontinue study drug in subjects with Grade 3-4 infusion reactions.

Concomitant Therapy

All concomitant medications, blood products, and radiotherapy administered will be recorded from Day 1 (predose) through the safety reporting period. Any concomitant medication given for a study protocol-related AE should be recorded from the time of informed consent.

Permitted Concomitant Therapy

Subjects may continue to use any ongoing medications not prohibited by the inclusion/exclusion criteria. However, efforts should be made to maintain stable doses of concomitant medications during the course of study treatment. All blood products and concomitant medications received from the first day of study treatment administration until 30 days after the final dose of any study drug are to be recorded in the medical records.

Screening/Baseline Assessments

Screening/Baseline assessments will be conducted to establish study baseline status and determine study eligibility. Only subjects who meet all inclusion and exclusion criteria specified in Section 4 will be enrolled in this study.

Subject medical history includes a thorough review of significant past medical history, current conditions, any treatment for prior malignancies and response to prior treatment, and any concomitant medications.

A physical exam, height, vital signs, weight, disease assessment (CT, PET-CT, or MRI scan) for baseline response efficacy assessment, confirmation of last blood draw, CBC with differential, urinalysis, ECHO/MUGA, hepatitis B and C screening, serum chemistry panel, serum cystatin C, coagulation tests, ECOG performance status, ECG, blood sample for biomarker analysis, collection of archival lesion biopsy (if available), and serum pregnancy test (for females of childbearing potential) are required for all subjects at screening and/or baseline.

Pharmacokinetic Assessments

Individual tucatinib plasma and oxaliplatin plasma (total and free platinum) and urine (platinum) concentrations at each sampling time will be listed and summarized with descriptive statistics.

PK parameters for tucatinib and oxaliplatin to be calculated (if possible) may include but are not limited to: AUClast, Cmax, Tmax, and Ctrough.

Additional exploratory PK analyses may be conducted, including exploratory analyses investigating the relationship between tucatinib and oxaliplatin exposure, efficacy and safety endpoints.

Biomarker Studies

Relationships of biomarker parameters (e.g., baseline values, absolute and relative changes from baseline) to efficacy, safety, and PK parameters may be explored. Relationships and associated data that are determined to be of interest will be summarized. Details will be described separately in the SAP or biomarker analysis plan.

Adverse Events

An overview of AEs will provide a tabulation of the incidence of all AEs, treatment emergent AEs, treatment-related AEs, Grade 3 and higher AEs, SAEs, treatment-related SAEs, deaths, and AEs leading to study treatment discontinuation. AEs will be defined as treatment emergent if they are newly occurring or worsen following study treatment.

AEs will be listed and summarized by MedDRA preferred term, severity, and relationship to study drug. In the event of multiple occurrences of the same AE with the same preferred term in 1 subject, the AE will be counted once as the occurrence. The incidence of AEs will be tabulated by preferred term and treatment group. AEs leading to premature discontinuation of study drug will be summarized and listed in the same manner.

All collected AE data will be listed by treatment group, study site, subject number, and cycle. Separately, all serious AEs and AEs of special interest (e.g., any DILI, asymptomatic left ventricular systolic dysfunction, and/or cerebral edema) will be analogously listed.

### Example 2: Combination of Tucatinib and Trastuzumab in Colorectal Cancer PDX Models

In this example, the efficacy of tucatinib and trastuzumab was evaluated in PDX models of HER2 positive CRC. Mice were subcutaneously inoculated with CTG-0121, CTG-0784, or CTG-0383 cells, and subsequently treated with tucatinib, trastuzumab, or a combination of the two drugs (n=10 per group). Tucatinib was administered orally at a dose of 50 mg/kg twice per day for 28 days (study days 0-27). Trastuzumab was administered intraperitoneally at a dose of 20 mg/kg once every three days. Nine doses of trastuzumab were administered, starting on study day 0. A vehicle-only group was included as a negative control.

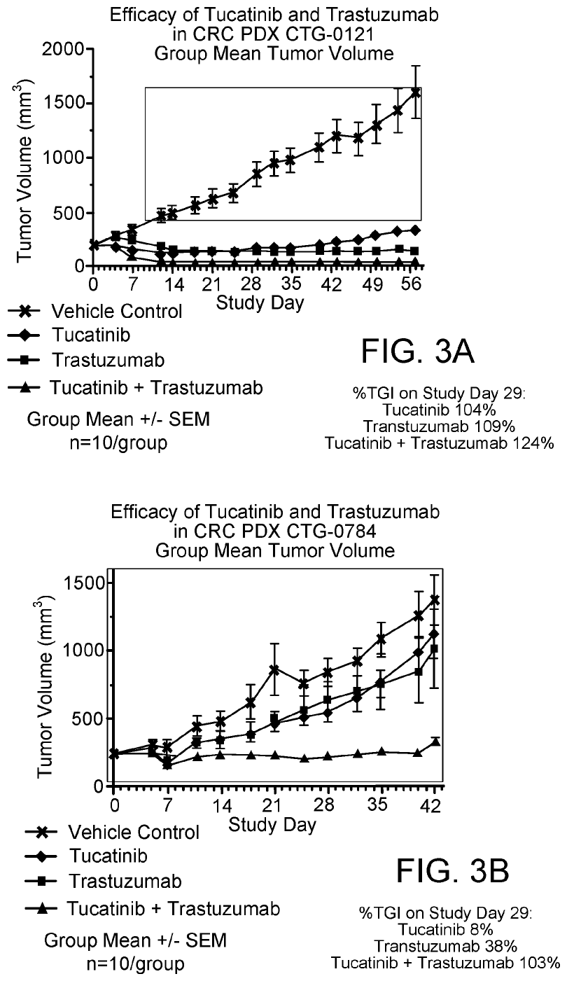

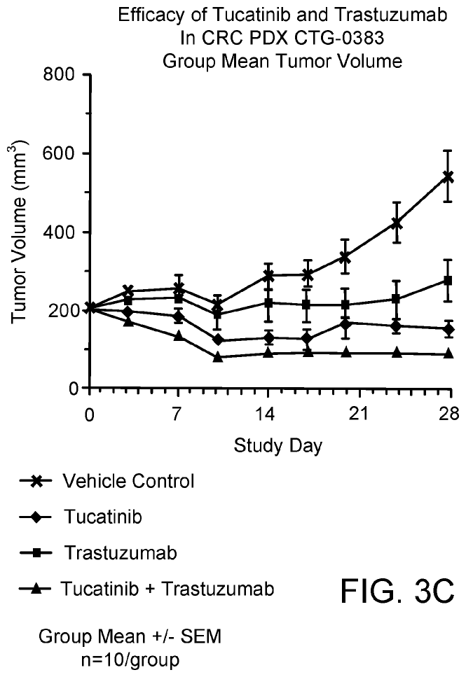

As shown in FIGS. 3A-3C, both tucatinib and trastuzumab inhibited tumor growth in all three CRC PDX models. Furthermore, when a combination of the two drugs was administered, the inhibition of tumor growth was more pronounced than when either drug was used individually. In the CTG-0121 model, tucatinib, trastuzumab, and a combination of the two drugs produced tumor growth inhibition (TGI) indices of 104%, 109%, and 124%, respectively, at study day 29 (Table 8). In the CTG-0784 model, tucatinib, trastuzumab, and a combination of the two drugs produced TGI indices of 50%, 36%, and 103%, respectively, at study day 29. In the CTG-0383 model, tucatinib, trastuzumab, and a combination of the two drugs produced TGI indices of 117%, 80%, and 137%, respectively, at study day 29. Surprisingly, a synergistic effect was observed when a combination of the two drugs was administered in all three models. Of note, the activity of a combination of tucatinib and trastuzumab in each HER2 positive CRC PDX model was comparable to activity observed in a HER2 positive breast cancer model (BT-474).

### Example 3: Combination of Tucatinib and Trastuzumab in Esophageal Cancer PDX Model

In this example, the efficacy of tucatinib and trastuzumab was evaluated in PDX models of HER2 positive esophageal cancer. Mice were subcutaneously inoculated with CTG-0137 or CTG-0138 cells, and subsequently treated with tucatinib, trastuzumab, or a combination of the two drugs (n=10 per group). Tucatinib was administered orally at a dose of 50 mg/kg twice per day for 28 days (study days 0-27). Trastuzumab was administered intraperitoneally at a dose of 20 mg/kg once every three days. Nine doses of trastuzumab were administered, starting on study day 0. A vehicle-only group was included as a negative control.

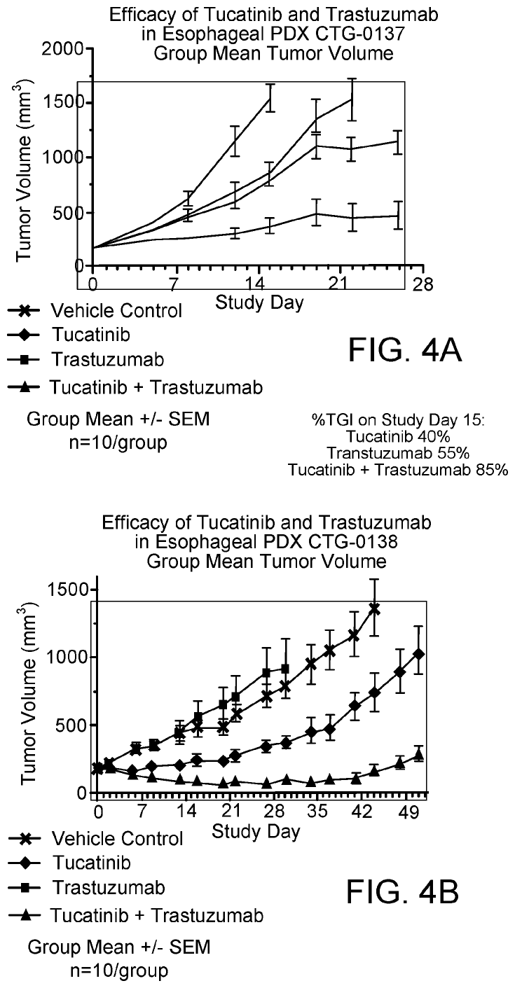

In the CTG-0137 model, both tucatinib and trastuzumab inhibited tumor growth, exhibiting TGI indices at study day 15 of 49% and 55%, respectively (FIG. 4A and Table 8). Furthermore, a synergistic effect was observed when a combination of the two drugs was administered, producing a TGI index of 85%.

In the CTG-0138 model, tucatinib inhibited tumor growth when administered as a single agent, producing a TGI index of 69% at study day 30 (FIG. 4B). However, a synergistic effect was observed when tucatinib and trastuzumab were administered in combination, producing a TGI index of 120% (Table 8).

### Example 4: Combination of Tucatinib and Trastuzumab in Gastric Cancer PDX Models

In this example, the efficacy of tucatinib and trastuzumab was evaluated in PDX models of HER2 positive gastric cancer. Mice were subcutaneously inoculated with GXA 3038, GXA 3039, or GXA 3054 cells, and subsequently treated with tucatinib, trastuzumab, or a combination of the two drugs (n=10 per group). Tucatinib was administered orally at a dose of 50 mg/kg twice per day for 28 days (study days 0-27). Trastuzumab was administered intraperitoneally at a dose of 20 mg/kg once every three days. Nine doses of trastuzumab were administered, starting on study day 0. A vehicle-only group was included as a negative control.

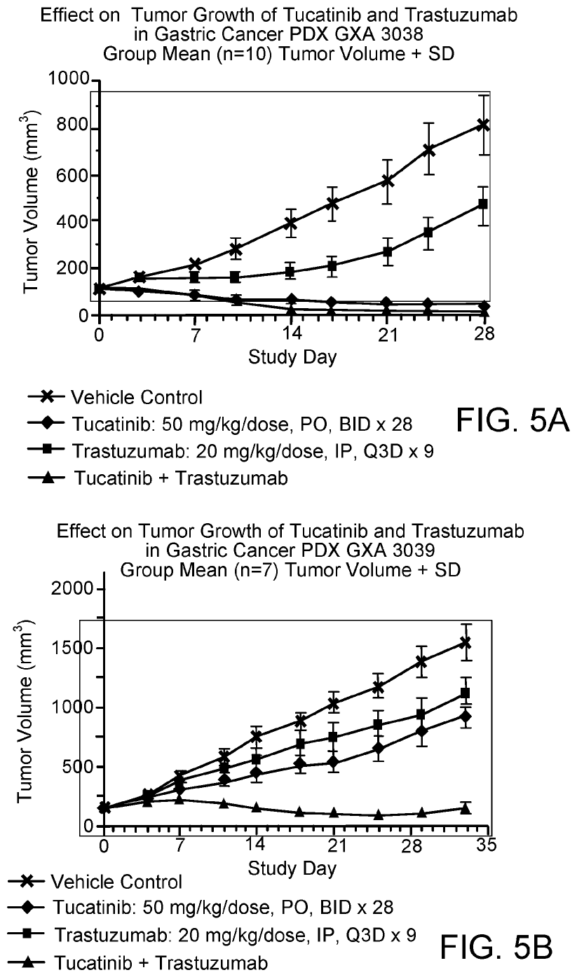

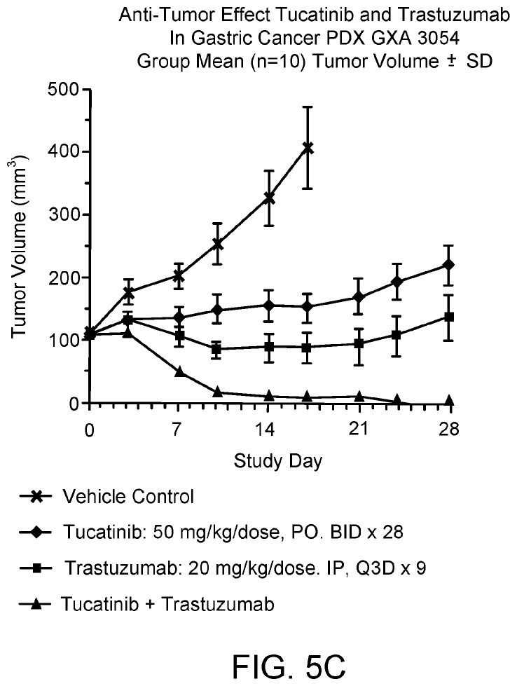

As shown in FIGS. 5A-5C, both tucatinib and trastuzumab inhibited tumor growth in all three gastric cancer PDX models. Furthermore, when a combination of the two drugs was administered, the inhibition of tumor growth was more pronounced than when either drug was used individually. In the GXA-3038 model, tucatinib, trastuzumab, and a combination of the two drugs produced TGI indices of 110%, 50%, and 116%, respectively, at study day 28 (Table 8). In the GXA-3039 model, tucatinib, trastuzumab, and a combination of the two drugs produced TGI indices of 48%, 38%, and 103%, respectively, at study day 29. In the GXA-3054 model, tucatinib, trastuzumab, and a combination of the two drugs produced TGI indices of 65%, 93%, and 136%, respectively, at study day 17. Surprisingly, a synergistic effect was observed when a combination of the two drugs was administered in all three models.

### Example 5: Combination of Tucatinib and Trastuzumab in a Cholangiocarcinoma PDX Model

In this example, the efficacy of tucatinib and trastuzumab was evaluated in a PDX model of HER2 positive cholangiocarcinoma. Mice were subcutaneously inoculated with CTG-0927 cells and subsequently treated with tucatinib, trastuzumab, or a combination of the two drugs (n=10 per group). Tucatinib was administered orally at a dose of 50 mg/kg twice per day for 28 days (study days 0-27). Trastuzumab was administered intraperitoneally at a dose of 20 mg/kg once every three days. Nine doses of trastuzumab were administered, starting on study day 0. A vehicle-only group was included as a negative control.

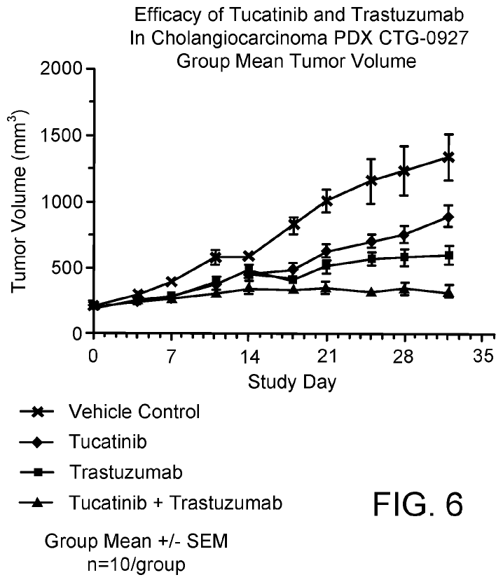

As shown in FIG. 6 and Table 8, both tucatinib and trastuzumab inhibited tumor growth. Furthermore, when a combination of the two drugs was administered, the inhibition of tumor growth was more pronounced than when either drug was used individually. At study day 28, the TGI indices for the tucatinib, trastuzumab, and combination therapy groups were 48%, 63%, and 86%, respectively.

### Example 6: Combination of Tucatinib and Trastuzumab in NSCLC Model

In this example, the efficacy of tucatinib and trastuzumab was evaluated in two different models of HER2 positive NSCLC. For these two studies, Calu-3 and NCI-H2170 cells were used, both of which express high levels of HER2, have gene amplification comparable to that of BT-474 breast cancer cells, and have previously demonstrated good responses to tucatinib in vitro.

Mice were subcutaneously inoculated with Calu-3 or NCI-H2170 cells and subsequently treated with tucatinib, trastuzumab, or a combination of the two drugs (n=10 per group). For the Calu-3 study, tucatinib was administered orally at a dose of 50 mg/kg twice per day for 21 days, beginning on study day 7. Trastuzumab was administered intraperitoneally at a dose of 20 mg/kg once every three days, beginning on study day 7. Seven doses of trastuzumab were administered. A vehicle-only group was included as a negative control. Three individual animals received dose holidays (one in the negative control group and two in the combination therapy group).

For the NCI-H2170 study, tucatinib was administered orally at a dose of 50 mg/kg twice per day for 21 days, beginning on study day 18. Trastuzumab was administered intraperitoneally at a dose of 20 mg/kg twice per week, beginning on study day 18. A vehicle-only group was included as a negative control.

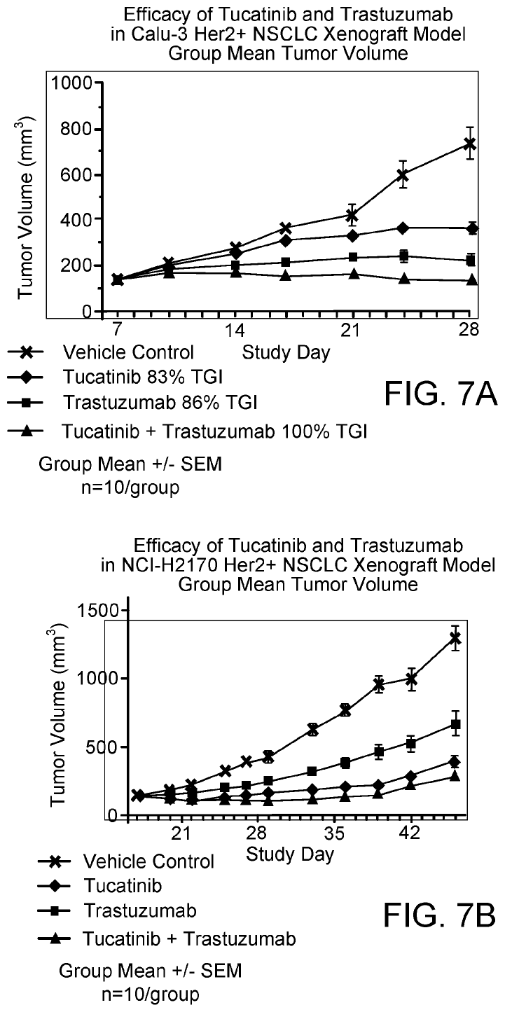

As shown in FIGS. 7A and 7B and Table 8, both tucatinib and trastuzumab inhibited tumor growth in both NSCLC models. Furthermore, when a combination of the two drugs was administered, the inhibition of tumor growth was more pronounced than when either drug was used individually. For the Calu-3 model, tucatinib, trastuzumab, and a combination of the two drugs produced tumor growth inhibition (TGI) indices of 63%, 86%, and 100%, respectively, at study day 28. Surprisingly, a synergistic effect was observed in the combination therapy group. For the NCI-2170 model, tucatinib, trastuzumab, and a combination of the two drugs produced TGI indices of 91%, 61%, and 98%, respectively, at study day 39.

TABLE 8

| | | | | Observed TGI (%) | | | Predicted % TGI |
|---|---|---|---|---|---|---|---|
| | | Type of | | | | Tucatinib + | Tucatinib + |
| Tumor name | Cancer Type | Xenograft | Vendor | Tucatinib | Trastuzumab | Trastuzumab | Trastuzumab |
| Calu-3 | NSCLC | CDX | BioDuro | 63 | 86 | 100 | 95 |
| NCH-H2170 | NSCLC | CDX | In house | 91 | 61 | 98 | 97 |
| CTG-0121 | CRC | PDX | Champions | 104 | 109 | 124 | 100 |
| | | | Oncology | | | | |
| CTG-0784 | CRC | PDX | Champions | 50 | 36 | 103 | 68 |
| | | | Oncology | | | | |
| CTG-0383 | CRC | PDX | Champions | 117 | 80 | 137 | 103 |
| | | | Oncology | | | | |
| CTG-0137 | Esophageal | PDX | Champions | 49 | 55 | 85 | 77 |
| | | | Oncology | | | | |
| CTG-0138 | Esophageal | PDX | Champions | 69 | −34 | 120 | 59 |
| | | | Oncology | | | | |
| CTG-0927 | Cholangio- | PDX | Champions | 48 | 63 | 86 | 81 |
| | carcinoma | | Oncology | | | | |
| GXA-3038 | Gastric | PDX | Oncotest | 110 | 50 | 116 | 105 |
| | carcinoma | | | | | | |
| | (Asian) | | | | | | |
| GXA-3039 | Gastric | PDX | Oncotest | 48 | 38 | 103 | 68 |
| | carcinoma | | | | | | |
| | (Asian) | | | | | | |
| GXA-3054 | Gastric | PDX | Oncotest | 65 | 93 | 136 | 98 |
| | carcinoma | | | | | | |
| | (Asian) | | | | | | |

1. A method of treating a HER2 positive cancer in a subject in need thereof, the method comprising administering to the subject a therapeutically effective amount of a combination therapy comprising tucatinib, trastuzumab and an oxaliplatin-based chemotherapy.

2. A method of treating cancer in a subject in need thereof, the method comprising:
(a) identifying the subject as having a HER2 positive cancer; and
(b) administering to the subject a therapeutically effective amount of a combination therapy comprising tucatinib, trastuzumab, and an oxaliplatin-based chemotherapy.

3. The method of any one of claim 1 or 2, wherein the oxaliplatin-based chemotherapy is oxaliplatin in combination with a compound selected from the group consisting of leucovorin (LV), fluorouracil, uracil-tegafur (UFT), irinotecan, and bevacizumab, or a combination thereof.

4. The method of any one of claims 1-3 wherein the oxaliplatin-based chemotherapy is administered as a regimen selected from the group consisting of FOLFOX4, mFOLFOX4, FOLFOX6, mFOLFOX6, mFOLFOX7, FOLFOXIRI, bFOL, PVIFOX, IROX, FUOX, FuFOX, CapeOx, XELOX, and CAPOX.

5. The method of any one of claims 1-4, wherein the oxaliplatin-based chemotherapy comprises oxaliplatin, leucovorin, and fluorouracil administered as an mFOLFOX7 regimen.

6. The method of any one of claims 1-5, wherein the HER2 positive cancer is selected from the group consisting of gastric adenocarcinoma, gastroesophageal junction (GEJ) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, brain cancer, and breast cancer

7. The method of any one of claims 1-6, wherein the HER2 positive cancer is gastric adenocarcinoma.

8. The method of any one of claims 1-6, wherein the HER2 positive cancer is gastroesophageal junction (GEJ) adenocarcinoma.

9. The method of any one of claims 1-6, wherein the HER2 positive cancer is esophageal adenocarcinoma.

10. The method of any one of claims 1-6, wherein the HER2 positive cancer is colorectal carcinoma (CRC).

11. The method of any one of claims 1-6, wherein the HER2 positive cancer is cholangiocarcinoma.

12. The method of any one of claims 1-6, wherein the HER2 positive cancer is gallbladder carcinoma.

13. The method of any one of claims 1-12, wherein the HER2 positive cancer is unresectable or metastatic.

14. The method of any one of claims 1-13, wherein the subject has not been previously treated with tucatinib.

15. The method of any one of claims 1-14, wherein the subject has not been previously treated with trastuzumab.

16. The method of any one of claims 1-15, wherein the subject has not been previously treated with an oxaliplatin-based chemotherapy.

17. The method of any one of claims 1-16, wherein the subject is a candidate to receive an oxaliplatin-based chemotherapy.

18. The method of any one of claims 1-15, wherein the subject was previously treated or is currently being treated with an oxaliplatin-based chemotherapy.

19. The method of any one of claims 1-18, wherein the subject was previously treated with at least one anticancer therapy.

20. The method of claim 19, wherein the least one anticancer therapy is an anti-HER2 antibody or an anti-HER2 antibody-drug conjugate.

21. The method of claim 19, wherein the at least one anticancer therapy is selected from the group consisting of trastuzumab, trastuzumab and a taxane, pertuzumab, ado-trastuzumab (T-DM1), and combinations thereof.

22. The method of any one of claims 19-21, wherein the subject is refractory to the at least one anticancer therapy.

23. The method of any one of claims 19-22, wherein the subject developed a brain metastasis during the previous treatment with the at least one anticancer therapy.

24. The method of any one of claims 1-23, wherein the subject has not been treated with another anticancer therapy within the past 12 months.

25. The method of any one of claims 1-24, wherein the tucatinib is administered to the subject at a dose of about 100 mg to about 1000 mg.

26. The method of any one of claims 1-25, wherein the tucatinib is administered twice daily.

27. The method of any one of claims 1-26, wherein the tucatinib is administered to the subject orally.

28. The method of any one of claims 1-27, wherein the trastuzumab is administered to the subject at a dose of about 6 mg/kg.

29. The method of any one of claims 1-28, wherein the trastuzumab is administered to the subject at a dose of about 4 mg/kg.

30. A method for treating a HER2 positive cancer in a subject that has exhibited an adverse event after starting treatment with a combination therapy comprising tucatinib, trastuzumab, and an oxaliplatin-based chemotherapy at an initial dosage level, comprising administering to the subject the at least one component of the combination therapy at a reduced dosage level.

31. The method of claim 30, wherein the tucatinib is administered to the subject at a dose of about 100 mg to about 1000 mg.

32. The method of any one of claim 30 or 31, wherein the trastuzumab is administered to the subject at a dose of about 6 mg/kg.

33. The method of any one of claim 30 or 31, wherein the trastuzumab is administered to the subject at a dose of about 4 mg/kg.

34. A kit for treating or ameliorating the effects of a HER2 positive cancer in a subject, the kit comprising tucatinib, trastuzumab, and an oxaliplatin-based chemotherapy.

35. A method of treating a HER2 positive cancer in a subject in need thereof, the method comprising:
(a) administering to the subject a therapeutically effective amount of a combination therapy comprising tucatinib, trastuzumab, and an oxaliplatin-based chemotherapy; and
(b) administering an effective amount of an anti-diarrheal agent.

36. A method of reducing the severity or incidents of diarrhea, or preventing diarrhea in a subject having a HER2 positive cancer and being treated with an effective amount of a combination therapy comprising tucatinib, trastuzumab, and an oxaliplatin-based chemotherapy, the method comprising administering an effective amount of an anti-diarrheal agent prophylactically.

37. A method of reducing the likelihood of a subject developing diarrhea, wherein the subject has a HER2 positive cancer and is being treated with an effective amount of a combination therapy comprising tucatinib, trastuzumab, and an oxaliplatin-based chemotherapy the method comprising administering an effective amount of an anti-diarrheal agent prophylactically.

38. The method of any one of claims 36-37, wherein the combination therapy and the anti-diarrheal agent are administered concurrently.

39. The method of any one of claims 36-37, wherein the anti-diarrheal agent is administered prior to administration of the combination therapy.

40. The method of any one of claims 36-39, wherein the subject is exhibiting symptoms of diarrhea.

41. The method of any one of claims 36-39, wherein the subject is not exhibiting symptoms of diarrhea.


SEQUENCE LISTING (4 sequences)

SEQ ID NO: 1 - protein, 449 aa, Artificial Sequence
  synthetically generated peptides
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Artificial Sequence
  synthetically generated peptides
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 108 aa, Artificial Sequence
  synthetically generated peptides
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQTKVEIKRT

SEQ ID NO: 4 - protein, 120 aa, Artificial Sequence
  synthetically generated peptides
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS